SEQUENCE LISTING (14 sequences)

SEQ ID NO: 1 - protein, 80 aa, Homo sapiens
PTLTLEPVNIMELYLGAKESKSFTFYRRDMGLTSSFESAAYPGWFLCTVPEADQPVRLTQLPENGGWNAPITDFYFQQCD

SEQ ID NO: 2 - DNA, 1282 bp, Homo sapiens
  CDS (73)...(537)
ccacgcgtccgcacagctcccgccaggagaaaggaacattctgaggggagtctacaccctgtggagctcaagatggtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgccccccagaactccctgggcagagccagctcgggtgaggggtgagtggaggagacccatggcggacaatcactctctctgctctcaggacccccacgtctgacttagtgggcacctgaccactttgtcttctggttcccagtttggataaattctgagatttggagctcagtccacggtcctcccccactggatggtgctactgctgtggaaccttgtaaaaaccatgtggggtaaactgggaataacatgaaaagatttctgtgggggtggggtgggggagtggtgggaatcattcctgcttaatggtaactgacaagtgttaccctgagccccgcaggccaacccatccccagttgagccttatagggtcagtagctctccacatgaagtcctgtcactcaccactgtgcaggagagggaggtggtcatagagtcagggatctatggcccttggcccagccccacccccttccctttaatcctgccactgtcatatgctacctttcctatctcttccctcatcatcttgttgtgggcatgaggaggtggtgatgtcagaagaaatggctcgagctcagaagataaaagataagtagggtatgctgatcctcttttaaaaacccaagatacaatcaaaatcccagatgctggtctctattcccatgaaaaagtgctcatgacatattgagaagacctacttacaaagtggcatatatttgcaattaatttta

SEQ ID NO: 3 - protein, 155 aa, Homo sapiens
MVLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 4 - DNA, 2648 bp, Homo sapiens
cacagctcccgccaggagaaaggaacattctgaggggagtctacaccctgtggagctcaagatggtcctgagtggggcgctgtgcttccgaatgaaggactcggcattgaaggtgctttatctgcataataaccagcttctagctggagggctgcatgcagggaaggtcattaaaggtgaagagatcagcgtggtccccaatcggtggctggatgccagcctgtcccccgtcatcctgggtgtccagggtggaagccagtgcctgtcatgtggggtggggcaggagccgactctaacactagagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgccccccagaactccctgggcagagccagctcgggtgaggggtgagtggaggagacccatggcggacaatcactctctctgctctcaggacccccacgtctgacttagtgggcacctgaccactttgtcttctggttcccagtttggataaattctgagatttggagctcagtccacggtcctcccccactggatggtgctactgctgtggaaccttgtaaaaaccatgtggggtaaactgggaataacatgaaaagatttctgtgggggtggggtgggggagtggtgggaatcattcctgcttaatggtaactgacaagtgttaccctgagccccgcaggccaacccatccccagttgagccttatagggtcagtagctctccacatgaagtcctgtcactcaccactgtgcaggagagggaggtggtcatagagtcagggatctatggcccttggcccagccccacccccttccctttaatcctgccactgtcatatgctacctttcctatctcttccctcatcatcttgttgtgggcatgaggaggtggtgatgtcagaagaaatggctcgagctcagaagataaaagataagtagggtatgctgatcctcttttaaaaacccaagatacaatcaaaatcccagatgctggtctctattcccatgaaaaagtgctcatgacatattgagaagacctacttacaaagtggcatatatttgcaattaattttaattaaaagatacctatttatatatttctttatagaaaaaagtctggaagagtttacttcaattgtagcaatgtcagggtggtggcagtataggtgatttttcttttaattctgttaatttatctgtatttcctaatttttctacaatgaagatgaattccttgtataaaaataagaaaagaaattaatcttgaggtaagcagagcagacatcatctctgattgtcctcagcctccacttccccagagtaaattcaaattgaatcgagctctgctgctctggttggttgtagtagtgatcaggaaacagatctcagcaaagccactgaggaggaggctgtgctgaagttgtgtggctggaatctctgggtaaggaacttaaagaacaaaaatcatctggtaattctttcctagaaggatcacagcccctgggattccaaggcattggatccagtctctaagaaggctgctgtactggttgaattgtgtccccctcaaattcacatccttcttggaatctcagtctgtgagtttatttggagataaggtctctgcagatgtagttagttaagacaaggtcatgctggatgaaggtagacctaaattcaatatgactggtttccttgtatgaaaaggagaggacacagagacagaggagacgcggggaagactatgtaaagatgaaggcagagatcggagttttgcagccacaagctaagaaacaccaaggattgtggcaaccatcagaagcttggaagaggcaaagaagaattcttccctagaggctttagagggataacggctctgctgaaaccttaatctcagacttccagcctcctgaacgaagaaagaataaatttcggctgttttaagccaccaaggataattggttacagcagctctaggaaactaatacagctgctaaaatgatccctgtctcctcgtgtttacattctgtgtgtgtcccctcccacaatgtaccaaagttgtctttgtgaccaatagaatatggcagaagtgatggcatgccacttccaagattaggttataaaagacactgcagcttctacttgagccctctctctctgccacccaccgcccccaatctatcttggctcactcgctctgggggaagctagctgccatgctatgagcaggcctataaagagacttacgtggtaaaaaatgaagtctcctgcccacagccacattagtgaacctagaagcagagactctgtgagataatcgatgtttgttgttttaaagttgctcagttttggtctaacttgttatgcagcaatagataaataatatgcagagaaagagaaaaaaaaaaaaaaaaaaaaaaaaaaaaaaaaaaaaaaaaaaaaaaaaaaaaa

SEQ ID NO: 5 - DNA, 5751 bp, Homo sapiens
  misc_feature (2): n = A,T,C or G
cngttaatcctaccactatgagnatgtcttatcatttagtangaagatttttgctttttgcacatgaaaaataggttggaaaaagtataggttttgtgatctgtgtatgaaagctgtctatagtacatgtgtatgtgtgggagaaaaagtgttgtcattggttttctratgantcactmagaaaagcaagtattcacattttttcttgtggctgtctgattttcaggtttttctacaatgacatgtaggctgancattccctargcagagagtcccacytctaacatctcytgtaggcctggcaakgcagcagaaasncagaggaaggaaggaggragaaggaaggagtgaagraaggagtaaaaaggtaaggaagaaagggaatagggragraagggagraaatgggaagggaaaraaggaaaggaaggaaagagggagggaagaaagraagggaaaagggagggagtgagtgaatgaaagatggaaagaagraagaaagggagggaggcagggaggaaagaaagttgcgcttcccttgasctgccatgggcactgmytcttagggtctgaaagcccctgagatgcaaaagcctagtgctcacaaagagctggaaagcctcaaggaagttcttcaatatttctggaaggaaactgtctccagaagcttccctccccacgacagataatgagcagcaagtgcttctggcgacttagggtgatgtgaaatcacgctgggaatcctgctcctcctcaggtcctggcagtttcagggcccctccctaggccttacttaaaaggctgaggcatccttggaggaacaggcagactccacagctcccgccaggagaaaggaacattctgaggtatgctctggggcgctggtggtaccggagctctctcctgaccccagacccagaatctgctccgtggaggctgttcacatgctggggagctcggtgcagctgcttgctccccagaccccagccaactcagcctctctctccatgattttctgttgtttattccaaaataggggagtctacaccctgtggagctcaagatggtcctgagtggggcgctgtgcttccggtgagtgtatgaggccctggtttggtggtgtcctccggaggaagtgagttctggatagacccgttgtccagctctgagcaggagggaggaagggaggggctgccattgsrgctgkbaaattgtgaccagcacctcattgctcttagagttttcccagcctttttcaaataggggcaggactggggcargccatctcacaaggggwccctgatgctgagggggacaagtgaacctcccagwctaragctccagccaagtctatccaaggtgggaacgggggccaggatccctgctcagagctccgccattgtcccccatcacagtgaatggatgtaagctcacccactctgtgcccctacctycctgctactctttggggataatwataaaacaaaaaccattaccatcagccagtytgtmcacccactggcatgtaccaagccagacactctgccgtgttctgggcttaacaacagaggatgaaragtgggcctttctctcagkctaataaagvacttcccacgatgkgttctatgggactcgattagaggagkccccagaggcatccaggagatgctttacacagkggagctctctgatcaagtaaawgcagggaatwctgctttctacatcctctcataagagaaccacagcccagctcagcatatgagwgactgaggktttctgaagkaaggcaacttgttgaatcgyattdagctatgcatcgacccaatttttacactgcatccttttcccccatataacttttggagaaacccactttaggatacatcttccacctcataggatgccaggaaatcaactgagttcaaagatgagaaacaactttgaaaagttaaataaaagaaatttaaatttaaagaamctcctcacttagtaaggaatatatgaccaaatagaaatmcatgtatcttgaagaattgaagaatcaggctttaacgtggaagaggcctggatgtwatccmccccatcatcttagtgtagcaatggggaggctmaracccagagtgggcgagagagttgtctcctgcgactcagcagcttggaggmatagatggggcaagatcctagggctstgactcaccgscagcttctcttccaacagtagatgggttgggacagaaaaggttaaatagggtsaagsakctwrccmcasaytccagtgkgagactgtgrggtcatcctccttgtagrgcatgakcccagcagggctgrgagacaargctgtgctgttacttctggctacagtagkaagaaagagagacaaaatgcytgagwycmgggggyycyctggatccagggcakgctgragtgtccaccctcctcctaatgtagtcctcwccccttcctgatgtttcagaatgaaggactcggcattgaaggtgctttatctgcataataaccagcttctagctggagggctgcatgcagggaaggtcattaaaggtyggtratgaaacatgacccactttcckkggkctctatacactctcaggggagggggcctgaagagggcttagaatagtcatacagattakcataggcctacrgagcccaggcattagggcaghacaaaccaggctctaagcaaaggcaaataaaatactacacctmtcagcaaagtgaagacacacgctctggggccacctgaagcttytgtgcagaagtgagaatgttttccaakakgcttgtcttgtyattcccttacaggtagatwtaggtcaagcattgcattccctgggagccagtaagtaccaaggagagaactaacgtagattctctataccttttttcccatatgggagwgggtttctgcctctccaccctgggtcccctctgctctctgaagatcctcagtcacttagagtggagggacccagagaacaggtggcattgwtggacctcctgcttgctcactmtgcmccatgcactgcaacaggtccctctstaaaatagttygcacctgcccacctggggcacccttgctgagcwcagatgccaggtagatccktcagctaggccatatgtgtatgygtgtgcttactggtgkatgdatgtgtgcatscaggcatatatgtgtrarcatatgtgtscatgcatgtatctgtatgtaaccatgtatgtgtragtgcagktatgtaggtatgascatgtgtgtgaatatgtatatgtgtscatgcatgtatctgtgcatgtatgatctgatgtatgtgggtggtgagggratgtacagagaggaatgagaccctcttttgctctcagcarcctcacagggtgtagaaagttgtccaamcaattccaaaggggggcttattaagacagggttcagaaaaaggcctgagacccaaggggcattaaaggagggggttgagtctattttgggttgtagaggcttgaagatttgmccctgaaytagagggtggagtggaggtggtacaatgtgcttccatgccttgatgtccactctgggccagtggacaggagaanccatgtmatgccagctgctragaagcctcccttctgcccagcctgggggcaggccgtctcacagcagtcytgtgccatagarsgcaggacarggaraaaagaaggaaaggcatccaggccytgcatctggcntttttcccacaggtgaagagatcaagcgtggtccccaatcggtggctggatgccagcctgtcccccgtcatcttgggtgtccagggtggaagccagtgcctgtcatgtggggtggggcaggagccgactcttaacactagaggtgagacttggggcatcctcactggggactcagccacagatgctgagcctactgaagccgggcagyccacagccytggtgctgtgggacaccctagcaggattctgttgatggcagctttgcctcctccmtaaggatcctgcccagccctccctctgcccctgcttctgccctcacctgacctcccctcctctgccggcagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgccccccagaactccctgggcagagccagctcgggtgaggggtgagtggaggagacccatggcggacaatcactctctctgctctcaggacccccacgtctgacttagtgggcacctgaccactttgtcttctggttcccagtttggataaattctgagatttggagctcagtccacggtcctcccccactggatggtgctactgctgtggaaccttgtaaaaaccatgtggggtaaactgggaataacatgaaaagatttctgtgggggtggggtgggggagtggtgggaatcattcctgcttaatggtaactgacaagtgttaccctgagccccgcaggccaacccatccccagttgagccttatagggtcagtagctctccacatgaagtcctgtcactcaccactgtgcaggagagggaggtggtcatagagtcagggatctatggcccttggcccagccccacccccttccctttaatcctgccactgtcatatgctacctttcctatctcttccctcatcatcttgttgtgggcatgaggaggtggtgatgtcagaagaaatggctcgagctcagaagataaaagataagtagggtatgctgatcctcttttaaaaacccaagatacaatcaaaatccccagatgctggtctctattcccatgaaaaagtgctcatgacatattgagaagacctacttacaaagtggcatatattgcaatttattttaattaaaagatacctatttatatatttctttatagaaaaaagtctggaagagtttacttcaattgtagcaatgtcagggtggtggcagtataggtgatwtttcttttaattctgttaatttatctgtatttcctaatttttctacaatgaagatgaattccttgtataaaaataagaaaagaaattaatcttgaggtaagcagagcagacatcatctctgatkgcctcagcctccacttccccagagtaaattcaaattgaatcgagctctgctgctctggttggttgtagtagtgatcaggaawcagatctcagcaaagccactgaggaggaggctgtgatgagtttgtgtggctggaatctctgggtaaggaacttaaagaacaaaaatcatctggtaattctttcctagaaggatcacagcccctgggattccaaggcattggatccagtctctaagaaggctgctgtactggttgaattgtgtccccctcaaattcacatccttcttggaatctcagtctgtgagtttatttggagataaggtctctgcagatgtagttagttaagacaaggtcatgctggatgaaggtagacctaaattyaatatgactggtttycttgtatgaaaaggagaggacacagagacagaggagacgcggggaagactatgtaaagatgaaggcagagatcggagttttgcagccacaa

SEQ ID NO: 6 - DNA, 7605 bp, Homo sapiens
aatattgacagtatgcacagtcatagtttcattttacttattatttatttatttattgaggcagaagtctagctctcttctgtcatccagctgggagtacagtggctcaatcttggctcaccgcaatctcacctccaggttcaagcaattctcacacctcagcctcctgagtagcttggattacaaatgtgcactacaacccggctaatttttgtgttttcagtaaagatggggttttgccatgttggccaggcttgtctcatttcatcttatttctactgtcattccatttggtcaatttgtaatttaataattttgtacaaggcagtttctacattaattttatttttctgaaaagtgtctatcatattagtggcattatgaaaatcgtagattattttctgtgttttgaacaacttgatattttatttattcttgaacacttgtaggacctcttggctcatatgtctgaataccatttctttaaaatgtacgctttaacatgtatacatatgtaacaaacctgcacattgtacacatgtatcctaaaacttaaagtataataataataataaaagaaaaaagaaaaataaaacttaaaaaaataaagtgtatgctttaacaataaattattaataaatagttatctgtgttattcaagatataattaaattgttttttgtgcactgacccttacctttatttcagatccatcacagagagtgcatggaggtatttaaactgcattactgtttcacaattaagtattatctttatcaattaggattcaagaaagatcacttacagaattatagatggcatgagctagattttactttctaaagaaataaccagatacatgagcaaagatgttaatacaaagatgtttgtcacaacatggttttcaatagcaaaaaaagagagaaaaatatataaaagacaaataacagtggataggtttcaataaataatgttacagtgatacagttaaatactatacagctattgaagcatgtcattattcatatttagtatggaaagatattttgctattttgctacatgaaaaaatgaggttggaaaaagtataggttttgtgaatctgttgtatgaaagctgtcttatagttacatgtgtatgtgtgtggaggaaaaagtgttgtcattggttttctgatgatgcactcagaaaagacaagtattcacattttttcttgtggctgatctggattttcaggtttttctacaatgaacatgtaggctgaacattccctaagcaggagagtcccacctctaacatctcctgtaggcctggcaatggcaggcaggaaagacagaggaaggaaggagggagaagggaaggagtgaaggaaggagtgaaaaaggtaaggaagaaagggaataggggaggaagggaggaaatgggaagggaaagaaggaaakgaagggaaagagggaggggaagaaaggaaggggaaaagggagggagtgagttgaatgaaagatggaaagaaggaagaaagggagggaggcagggaggaaagaaagttgcgcttcccttgagctgcccatgggcacctgactcttagggtctgaaaggcccctgagatgcaaaagcctagtgctcacaaagagctggaaagcctcaaggaagttcttcaatatttctggaaggaaactgtctccagaagcttccctccccacgacagataatgagcagcaagtgcttctggcgacttagggtgatgtgaaattcacgctgggaatcctgctcctcctcaggtcctggcaagtttcagggcccctccctaggccttacttaaaaggctgaggcatccttggaggaacaggcagactccacagctcccgccaggagaaaggaacattctgaggtatgctctggggcgctggtggtaccggagctctctcctgaccccagacccagaatctgctccgtggaggctgttcacatgctggggagctcggtgcagctgcttgctccccagaccccagccaactcagcctctctctccatgattttctgttgtttattccaaaataggggagtctacaccctgtggagctcaagatggtcctgagtggggcgctgtgcttccggtgagtgtatgaggccctggtttggtggtgtcctccggaggaagtgagttctggatagacccgttgtccagctctgagcaggagggaggaagggagggggctgccattgcagctgggaaattgtgaccagcacctcattgctcttagagttttcccagcctttttcaaataggggcaggactggggcaggccatctcacaaggggtccctgatgctgagggggacaagtgaacctcccagtctagagctccagccaagtctatccaaggtgggaacgggggccaggatccctgctcagagctccgccattgtcccccatcacagtgaatggatgtaagctcacccactctgtgcccctacctccctgctactctttgggggataataataaaacaaaaaccattaccatcagccaagtctgtccacccactggcatgtaccaagccagacactctgccgtgttctgggcttaacaaccagaggatgagagtggtcctttctctcagtctaataaagcacttcccacgatgtgttctatgggactcgattagaggagtcccacagaggcatccaggagatgctttacacagtggagctctctgatcaagtaaatgcagggaattctgctttctacatcctctcataagagaaccacagcccagctcagcatatgagtgactgaggktttctgaagtaaggcaacttgttgaatcgyatttagctatgcatcgacccaatttttacactgcatccttttcccccatataacttttggagaaacccactttaggatacatcttccacctcataggatgccaggaaatcaactgagttcaaagatgagaaacaactttgaaaagttaaataaaagaaatttaaatttaaagaaactcctcacttagtaaggaatatatgaccaaatagaaatacatgtatcttgaagaattgaagaatcaggctttaacgtggaagaggcctggatgttatccaacccatcatcttagtgtagcaatggggaggctcagacccaagagtgggcgagagagttgtctcctgcgactcagcagcattggaggcatagatggggcaagatcctagggctctgactcaccgagcagcttctcttccaacaggagatgggttggggcagaaaaggttgaatagggtgaaggagcaaaccacagactccagtgggagactgtggggtcatcctccttgtagggcatgagcccagcagggctgggagacaaggctgtgctgttacttctggcacagtaggaagaaagagagacaaaatgcctgagatcagggggttctctggatccagggcatgctggagtgtccaccctcctcctaatgtagtcctcaccccttcctgatgtttcagaatgaaggactcggcattgaaggtgctttatctgcataataaccagcttctagctggagggctgcatgcagggaaggtcattaaaggttggtgatgaaacatgacccactttccttggtctctatacactctcaggggagggggcctgaagagggcttagaatagtcatacagattagcataggcctacagagcccaggcattagggcagcacaaaccaggctctaagcaaaggcaaataaaatactacacctctcagcaaagtgaagacacacgctctggggccacctgaagcttctgtgcagaagtgagaatgttttccaagaggcttgtcttgtcattcccttacaggtagatwtaggtcaagcattgcattccctgggagccagtaagtaccaaggagagaactaacgtagattctctataccttttttcccatatgggagtgggtttctgcctctccaccctgggtcccctctgctctctgaagatcctcagtcacttagagtggagggacccagagaacaggtggcattgttggacctcctgcttgctcactctgccccatgcactgcaacaggtccctctctaaaatagttygcacctgcccacctggggcacccttgctgagcacagatgccaggtagatccttcagctaggccatatgtgtatgtgtgtgcttactggtgtatgtatgtgtgcatgcaggcatatatgtgtgagcatatgtgtgcatgcatgtatctgtatgtaaccatgtatgtgtgagtgcaggtatgtaggtatgagcatgtgtgtgaatatgtatatgtgtgcatgcatgtatctgtgcatgtatgatctgatgtatgtgggtggtgaggggatgtacagagaggaatgagaccctcttttgctctcagcaacctcacagggtgtagaaagttgtccaaacaattccaaaggggggcttattaagacagggttcagaaaaaggcctgagacccaaggggcattaaaggagggggttgagtctattttgggttgtagaggcttgaagatttgaccctgaactagagggtggagtggaggtggtacaatgtgcttccatgccttgatgtccactctgggccagtggacaggagaagccatgtcatgacagctgctgagaagcctcccttctgcccagcctgggggcaggccgtctcacagcagtcctgtgccctagagcccaggacaggggaagaaggagggaaaggcatccagggccctgcatctggcctctttcccacaggtgaagagatcagcgtggtccccaatcggtggctggatgccagcctgtcccccgtcatcctgggtgtccagggtggaagccagtgcctgtcatgtggggtggggcaggagccgactcttaacactagaggtgagacttggggcatcctcactggggactcagccacagatgctgagcctactgaagccgggcagcccacagccctggtgctgtgggacaccctagcaggattctgttgatggcagctttgcctcctccctaaggatcctgcccagccctccctctgcccctgcttctgccctcacctgacctcccctcctctgccggcagccagtgaacatcatggagctctatcttggtgccaaggaatccaagagcttcaccttctaccggcgggacatggggctcacctccagcttcgagtcggctgcctacccgggctggttcctgtgcacggtgcctgaagccgatcagcctgtcagactcacccagcttcccgagaatggtggctggaatgcccccatcacagacttctacttccagcagtgtgactagggcaacgtgccccccagaactccctgggcagagccagctcgggtgaggggtgagtggaggagacccatggcggacaatcactctctctgctctcaggacccccacgtctgacttagtgggcacctgaccactttgtcttctggttcccagtttggataaattctgagatttggagctcagtccacggtcctcccccactggatggtgctactgctgtggaaccttgtaaaaaccatgtggggtaaactgggaataacatgaaaagatttctgtgggggtggggtgggggagtggtgggaatcattcctgcttaatggtaactgacaagtgttaccctgagccccgcaggccaacccatccccagttgagccttatagggtcagtagctctccacatgaagtcctgtcactcaccactgtgcaggagagggaggtggtcatagagtcagggatctatggcccttggcccagccccacccccttccctttaatcctgccactgtcatatgctacctttcctatctcttccctcatcatcttgttgtgggcatgaggaggtggtgatgtcagaagaaatggctcgagctcagaagataaaagataagtagggtatgctgatcctcttttaaaaacccaagatacaatcaaaatcccagatgctggtctctattcccatgaaaaagtgctcatgacatattgagaagacctacttacaaagtggcatatattgcaatttattttaattaaaagatacctatttatatatttctttatagaaaaaagtctggaagagtttacttcaattgtagcaatgtcagggtggtggcagtataggtgatttttcttttaattctgttaatttatctgtatttcctaatttttctacaatgaagatgaattccttgtataaaaataagaaaagaaattaatcttgaggtaagcagagcagacatcatctctgattgcctcagcctccacttccccagagtaaattcaaattgaatcgagctctgctgctctggttggttgtagtagtgatcaggaatcagatctcagcaaagccactgaggaggaggctgtgatgagtttgtgtggctggaatctctgggtaaggaacttaaagaacaaaaatcatctggtaattctttcctagaaggatcacagcccctgggattccaaggcattggatccagtctctaagaaggctgctgtactggttgaattgtgtccccctcaaattcacatccttcttggaatctcagtctgtgagtttatttggagataaggtctctgcagatgtagttagttaagacaaggtcatgctggatgaaggtagacctaaattcaatatgactggtttccttgtatgaaaaggagaggacacagagacagaggagacgcggggaagactatgtaaagatgaaggcagagatcggagttttgcagccacaagctaagaaacaccaaggattgtggcaaccatcagaagcttggaagaggcaaagaagaattcttccctagaggctttagagggataacggctctgctgacaccttaatctcagacttccagcctcctgaacgaagaaagaataaatttcggctgttttaagccaccaaggataattggttatggcagctctaggaaactaatacagctgctaaaatgatccctgtctcctcgtgtttacattctgtgtgtgtcccctcccacaatgtaccaaagttgtctttgtgaccaatagaatatggcagaagtgatggcatgccacttccaagattaggttataaaagacactgcagcttctacttgagccctctctctctgccacccaccgcccccaatctatcttggctcactcgctctgggggaagctagcttccatgctatgagcaggcctataaagagacttatgtggtaaaaaatgaagtctcctgcccacagccacattagtgaacctagaagcagagactctgtgagataatcaatgtttgttgttttaagttgctcagttttggtctaacttgttatgcagcaatagataaataatatgcagagaaagagaaacaaatgcatttgttttattattgcaattttctccaatattttttattttctttctcacaatgaacaactatccttcatttacccaaatattctatttaaaagctaataatacagcatttgttgagtcatctggttctgcaagattgagatcctcttgtcctatgtgccaggaatgaactccagtgccccacccaaaccctggggaatg

SEQ ID NO: 7 - protein, 147 aa, Homo sapiens
FRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 8 - protein, 13 aa, Artificial Sequence
  Description of Artificial Sequence Peptide of IL-1Hy1
RLTQLPENGGWNA

SEQ ID NO: 9 - DNA, 20 bp, Artificial Sequence
  Description of Artificial Sequence Riboprobe
cacagctcccgccaggagaa

SEQ ID NO: 10 - DNA, 20 bp, Artificial Sequence
  Description of Artificial Sequence Riboprobe
gggaccacgctgatctcttc

SEQ ID NO: 11 - DNA, 20 bp, Artificial Sequence
  Description of Artificial Sequence Riboprobe
agcttcccgagaatggtggc

SEQ ID NO: 12 - DNA, 20 bp, Artificial Sequence
  Description of Artificial Sequence Riboprobe
gtggtcaggtgcccactaag

SEQ ID NO: 13 - DNA, 22 bp, Artificial Sequence
  Description of Artificial Sequence Riboprobe
ctgggtaaggaacttaaagaac

SEQ ID NO: 14 - DNA, 21 bp, Artificial Sequence
  Description of Artificial Sequence Riboprobe
tcttaactaactacatctgca

(19) United States
(12) Patent Application Publication
(10) Pub. No.: US 2003/0099650 A1
(43) Pub. Date: May 29, 2003

(54) Treatment of immune disorders and B cell disorders

(76) Inventors: Alice Suk-Yue Ho, Palo Alto, CA (US); John E. Ford, San Diego, CA (US); Hai-Shan Lin, Castro Valley, CA (US); Fabio Rupp, Sunnyvale, CA (US)

Correspondence Address: Li-Hsien Rin-Laures, HYSEQ, INC., 670 Almanor Avenue, Sunnyvale, CA, 94085, US

(21) Appl. No.: 10/205,821

(22) Filed: Jul. 25, 2002

Related U.S. Application Data
(60) Provisional application No. 60/307,754, filed on Jul. 25, 2001.
(60) Provisional application No. 60/334,668, filed on Nov. 29, 2001.

(51) Int. Cl. A61K 39/395

(52) U.S. Cl. 424/145.1

(57) ABSTRACT

The present invention provides materials and methods of using compositions comprising the IL-1Hy1 polypeptide or inhibitors of IL-1Hy1 activity for treatment of immune cell disorders.

### RELATED APPLICATIONS

This application claims priority benefit of U.S. Provisional Application No. 60/307,754 filed Jul. 25, 2001 and U.S. Provisional Application No. U.S. 60/334,668 filed Nov. 29, 2001, which are herein incorporated by reference in their entirety.

### FIELD OF THE INVENTION

The present invention provides novel polynucleotides and proteins encoded by such polynucleotides, along with therapeutic, diagnostic and research utilities for the treatment of immune cell and B cell related disorders for these polynucleotides and proteins.

### BACKGROUND

Mature B cells are derived from the bone marrow precursor cells and make up about 10-15% of the peripheral blood lymphocytes, 50% of the splenic lymphocytes and about 10% of the bone marrow lymphocytes. The primary function of the B cells is to produce antibodies.

B cell development, differentiation and proliferation is regulated by cytokines including IL-1. In particular, it is known that IL-7 drives pro-B and pre-pre B cell proliferation and differentiation. BCG-F (low-molecular weight B cell growth factor) and IL-1 induce B cell precursor proliferation. IL-1, IL-2, IL-4, IL-5 and IL-6 are known to induce mature B cell proliferation and to drive differentiation into antibody secreting cells.

B-cell development begins in the bone marrow and continues within the periphery. The bone marrow contains cells within the early stages of B-cell development (pro-B cells, pre-pre-B cells, pre-B cells, and immature B cells). The immature B cells develop into naïve B cells, which subsequently enter the periphery. Naïve cells develop into IgM-secreting lymphoblast cells, followed by memory B cells, and finally antibody-secreting plasma cells.

### SUMMARY OF THE INVENTION

The invention provides for methods of inhibiting B cell proliferation or B cell activation comprising administering an inhibitor of IL-1Hy1 activity to human with elevated B cell levels or B cell activity. Optionally, before, concurrently or after administration, B cell levels or activity may be measured in said human. The inhibitor of IL-1Hy1 activity may be for example, an antibody to IL-1Hy1, an antisense oligonucleotide, an inactive variant of IL-1Hy1 or a soluble form of a receptor that binds to IL-1Hy1, or a small molecule that inhibits binding of or activity of IL-1Hy1. The methods of inhibiting B cell proliferation can be an effective therapy for B cell related disorders such as B cell lymphoproliferative disorders (e.g., myelomas, lymphomas, leukemias), B cell related autoimmune diseases, allergy, asthma or allergic rhinitis. These methods of the invention optionally may be effective therapy for those B cell disorders not directly and/or primarily a result of IL-1β, IL-12 and/or IL-18 induced inflammation. The invention includes compounds for the preparation of medicaments useful for the inhibition of B cell proliferation in a human suffering from a B cell related disorder optionally those B cell related disorders not directly and/or primarily a result of IL-1 β, IL-12 and/or IL-18 induced inflammation. The invention also includes compositions comprising an inhibitor of IL-1Hy1 activity in an effective amount to inhibit B cell proliferation or B cell activity induced by IL-1Hy1. The compositions of the invention which inhibit B cell proliferation, differentiation and/or activation may be concurrently administered with a vaccine to improve efficacy of the vaccine.

The invention also provides for methods of stimulating B cell proliferation comprising administering an effective amount of IL-1Hy1, comprising the amino acid sequence of SEQ ID NO: 3 or a variant thereof, to a human suffering from a B cell immune deficiency or otherwise in need of higher B cell levels or activity (e.g., suffering from infection). Optionally, before, concurrently or after administration, B cell or antibody levels in said human may be measured. The invention includes compounds for the preparation of medicaments useful for the simulation of B cell proliferation in a human with B cell related disorders optionally those not directly and/or primarily a result of IL-1β, IL-12 and/or IL-18 induced inflammation. The invention also includes compositions comprising IL-1Hy1 polypeptide comprising the amino acid sequence of SEQ ID NO: 3 in an amount effective to stimulate B cell proliferation or activity. The compositions of the invention which stimulate B cell proliferation and/or activation may be concurrently administered with a vaccine to improve efficacy of the vaccine.

Also encompassed by the invention are methods for treating autoimmune diseases associated with increased production of IgA. These methods comprise administering an effective amount of IL-1Hy1, comprising the amino acid sequence of SEQ ID NO: 3 to a human suffering from a disorder related to elevated IgA levels. These disorders include but are not limited to IgA nephropathy, dermatitis herpetiformis and linear IgA disease. Optionally, before, concurrently or after the administration, IgA levels may be measured in said human. The invention includes compounds for the preparation of medicaments useful for the reduction of IgA production in a human with an IgA related disease optionally those not directly and/or primarily a result of IL-1β, IL-12 and/or IL-18 induced inflammation. The invention also includes compositions comprising an IL-1Hy1 polypeptide comprising the amino acid sequence of SEQ ID NO: 3 in an amount effective to reduce IgA production. The compositions of the invention which are useful for treating autoimmune diseases may be concurrently administered with a vaccine to improve efficacy of the vaccine.

Example 1 demonstrates that IL-1Hy1 polypeptide expression is elevated in allergic nasal polyps, chronically infected nasal tissue, and chronic lung tissue. This data suggests that IL-1Hy1 may play a role in modulating allergic reactions such as asthma and rhinitis. In addition, IL-1Hy1 polypeptide expression is elevated in ulcerative colitis and Crohn's Disease as described in Example 12 suggesting IL-1Hy1 modulates the inflammatory response in this tissue.

Treatment methods of the invention include administering an IL-1Hy1 polypeptide or polynucleotide of the invention or an inhibitor of the invention (e.g. an antibody to IL-1Hy1, an antisense oligonucleotide, an inactive variant of IL-1Hy1 or a soluble form of a receptor that binds to IL-1Hy1, or a small molecule that inhibits binding of or activity of IL-1Hy1).

The invention also provides for methods of altering B-cell differentiation. In embodiments wherein B-cell differentiation is stimulated, compositions comprising an effective amount of IL-1Hy1 activity or those activating an effective amount of IL-1Hy1 activity (e.g., increase the expression of IL-Hy1) are utilized. In preferred embodiments, the composition comprises an effective amount of an IL-1Hy1 polypeptide comprising the amino acid sequence of SEQ ID NO:3 or an active variant thereof. The compositions of the invention which are administered to alter B cell differentiation may be concurrently administered with a vaccine to improve efficacy of the vaccine. The method of stimulating differentiation can be an effective therapy against infections or immunoglobulin deficiency syndromes such as agammagglobilinema.

In embodiments wherein B-cell differentiation is inhibited, an inhibitor of IL-1Hy1 activity is utilized. The inhibitor of IL-1Hy1 activity may be for example, an antibody to IL-1Hy1, an antisense oligonucleotide, an inactive variant of IL-1Hy1 or a soluble form of a receptor that binds to IL-1Hy1, or a small molecule that inhibits binding of or activity of IL-1Hy1. The methods of inhibiting B cell differentiation can be an effective therapy for B cell-related disorders such as multiple myeloma and autoimmune diseases that result in autoantibody production such as systemic lupus erythematosus, Crohn's disease, graft-versus-host disease, and asthma; and optionally those B-cell related disorders and autoimmune diseases not directly and/or primarily a result of IL-1β, IL-12 and/or IL-18 induced inflammation.

Treatment methods of the invention include administering an IL-1Hy1 polypeptide or polynucleotide or an inhibitor of IL-1Hy1 (e.g., an antibody to IL-1 Hy1, an antisense oligonucleotide, an inactive variant of IL-1Hy1 or a soluble form of a receptor that binds to IL-1Hy1, or a small molecule that inhibits binding of or activity of IL-1Hy1) in an effective dose to alter B-cell differentiation.

### BRIEF DESCRIPTION OF SEQUENCES

SEQ ID NO: 1 represents a partial amino acid sequence of IL-Hy1 polypeptide.

SEQ ID NO: 2 represents the cDNA sequence which comprises the complete coding region that encodes the full length IL-1Hy1 polypeptide.

SEQ ID NO: 3 represents the full length amino acid sequence of IL-1Hy1 polypeptide.

SEQ ID NO: 4 represents a 5′ and 3′ extension of the polynucleotide sequence set out as SEQ ID NO: 2, and comprises the complete coding region that encodes the full length IL-1Hy1 polypeptide.

SEQ ID NO: 5 represents the genomic DNA sequence which comprises the coding region that encodes the full length IL-1Hy1 polypeptide.

SEQ ID NO: 6 represents a 3′ extension of the genomic sequence of SEQ ID NO: 5.

SEQ ID NO: 7 represents the an IL-1Hy1 amino acid sequence which is missing the first 5 amino acids as compared to SEQ ID NO: 3.

SEQ ID NO: 8 represents the peptide sequence used to generate the anti-IL-1Hy1 polyclonal antibody (Example 1).

### DETAILED DESCRIPTION

Definitions

The term “nucleotide sequence” refers to a heteropolymer of nucleotides or the sequence of these nucleotides. The terms “nucleic acid” and “polynucleotide” are also used interchangeably herein to refer to a heteropolymer of nucleotides. Generally, nucleic acid segments provided by this invention may be assembled from fragments of the genome and short oligonucleotide linkers, or from a series of oligonucleotides, or from individual nucleotides, to provide a synthetic nucleic acid which is capable of being expressed in a recombinant transcriptional unit comprising regulatory elements derived from a microbial or viral operon, or a eukaryotic gene.

The terms “oligonucleotide fragment” or a “polynucleotide fragment”, “portion,” or “segment” is a stretch of polypeptide nucleotide residues which is long enough to use in polymerase chain reaction (PCR) or various hybridization procedures to identify or amplify identical or related parts of mRNA or DNA molecules.

The terms “oligonucleotides” or “nucleic acid probes” are prepared based on the polynucleotide sequences provided in the present invention. Oligonucleotides comprise portions of such a polynucleotide sequence having at least about 15 nucleotides and usually at least about 20 nucleotides. Nucleic acid probes comprise portions of such a polynucleotide sequence having fewer nucleotides than about 6 kb, usually fewer than about 1 kb. After appropriate testing to eliminate false positives, these probes may, for example, be used to determine whether specific mRNA molecules are present in a cell or tissue or to isolate similar nucleic acid sequences from chromosomal DNA as described by Walsh et al. (Walsh, P. S. et al., 1992, PCR Methods Appl 1:241-250).

The term “probes” includes naturally occurring or recombinant or chemically synthesized single- or double-stranded nucleic acids. They may be labeled by nick translation, Klenow fill-in reaction, PCR or other methods well known in the art. Probes of the present invention, their preparation and/or labeling are elaborated in Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York N.Y., both of which are incorporated herein by reference in their entirety.

The term “stringent” is used to refer to conditions that are commonly understood in the art as stringent. Stringent conditions can include highly stringent conditions (i.e., hybridization to filter-bound DNA under in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.).

In instances wherein hybridization of deoxyoligonucleotides is concerned, additional exemplary stringent hybridization conditions include washing in 6×SSC/0.05% sodium pyrophosphate at 37° C. (for 14-base oligos), 48° C. (for 17-base oligos), 55° C. (for 20-base oligos), and 60° C. (for 23-base oligos).

The term “recombinant,” when used herein to refer to a polypeptide or protein, means that a polypeptide or protein is derived from recombinant (e.g., microbial or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

The term “recombinant expression system” means host cells that have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells that have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “open reading frame,” ORF, means a series of nucleotide triplets coding for amino acids without any termination codons and is a sequence translatable into protein.

The term “expression modulating fragment,” EMF, means a series of nucleotides which modulates the expression of an operably linked ORF or another EMF.

As used herein, a sequence is said to “modulate the expression of an operably linked sequence” when the expression of the sequence is altered by the presence of the EMF. EMFs include, but are not limited to, promoters, and promoter modulating sequences (including, e.g. inducible elements). One class of EMFs are fragments which induce the expression or an operably linked ORF in response to a specific regulatory factor or physiological event.

As used herein, an “uptake modulating fragment,” UMF, means a series of nucleotides that mediate the uptake of a linked DNA fragment into a cell. UMFs can be readily identified using known UMFs as a target sequence or target motif with the computer-based systems described below.

The presence and activity of a UMF can be confirmed by attaching the suspected UMF to a marker sequence. The resulting nucleic acid molecule is then incubated with an appropriate host under appropriate conditions and the uptake of the marker sequence is determined. As described above, a UMF will increase the frequency of uptake of a linked marker sequence.

The term “active” refers to those forms of the polypeptide that retain the biologic and/or immunologic activities of any naturally occurring polypeptide.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “derivative” refers to polypeptides chemically modified by such techniques as ubiquitination, labeling (e.g., with radionuclides or various enzymes), pegylation (derivatization with polyethylene glycol) and insertion or substitution by chemical synthesis of amino acids such as omithine, which do not normally occur in human proteins.

The term “recombinant variant” refers to any polypeptide differing from naturally occurring polypeptides by amino acid insertions, deletions, and substitutions, created using recombinant DNA techniques. Guidance in determining which amino acid residues may be replaced, added or deleted without abolishing activities of interest, such as cellular trafficking, may be found by comparing the sequence of the particular polypeptide with that of homologous peptides and minimizing the number of amino acid sequence changes made in regions of high homology.

Preferably, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

As used herein, “substantially equivalent” can refer both to nucleotide and amino acid sequences, for example a mutant sequence, that vary from a reference sequence by one or more substitutions, deletions, or additions, the net effect of which does not result in an adverse functional dissimilarity between the reference and subject sequences. Typically, such a substantially equivalent sequence varies from one of those listed herein by no more than about 20% (i.e., the number of individual residue substitutions, additions, and/or deletions in a substantially equivalent sequence, as compared to the corresponding reference sequence, divided by the total number of residues in the substantially equivalent sequence is about 0.2 or less). Such a sequence is said to have 80% sequence identity to the listed sequence. In one embodiment, a substantially equivalent, e.g., mutant, sequence of the invention varies from a listed sequence by no more than 10% (90% sequence identity); in a variation of this embodiment, by no more than 5% (95% sequence identity); and in a further variation of this embodiment, by no more than 2% (98% sequence identity). Substantially equivalent, e.g., mutant, amino acid sequences according to the invention generally have at least 95% sequence identity with a listed amino acid sequence, whereas substantially equivalent nucleotide sequence of the invention can have lower percent sequence identities, taking into account, for example, the redundancy or degeneracy of the genetic code. For the purposes of the present invention, sequences having substantially equivalent biological activity and substantially equivalent expression characteristics are considered substantially equivalent. For the purposes of determining equivalence, truncation of the mature sequence (e.g., via a mutation which creates a spurious stop codon) should be disregarded.

Nucleic acid sequences encoding such substantially equivalent sequences, e.g., sequences of the recited percent identities, can routinely be isolated and identified via standard hybridization procedures well known to those of skill in the art.

Where desired, an expression vector may be designed to contain a “signal or leader sequence” which will direct the polypeptide through the membrane of a cell. Such a sequence may be naturally present on the polypeptides of the present invention or provided from heterologous protein sources by recombinant DNA techniques.

A polypeptide “fragment,” “portion,” or “segment” is a stretch of amino acid residues of at least about 5 amino acids, often at least about 7 amino acids, typically at least about 9 to 13 amino acids, and, in various embodiments, at least about 17 or more amino acids. To be active, any polypeptide must have sufficient length to display biologic and/or immunologic activity. In a preferred embodiment, the IL-1Hy1 fragment has a molecular weight as determined by SDS-PAGE of about 16 kDa or less. More preferably, the IL-1Hy1 fragment has a molecular mass of about 15.5 kDa.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

The term “activated” cells as used in this application are those which are engaged in extracellular or intracellular membrane trafficking, including the export of neurosecretory or enzymatic molecules as part of a normal or disease process.

The term “differentiate” refers to the process by which B cells develop from stem cells into memory or plasma cells. Differentiation, and/or proliferation, and/or activation of B cells may be broken into distinct stages, e.g., Pro-B cell, pre-pre-B cells, pre-B cells, immature B cells, naïve B cells, lymphoblast cells, memory B cells, and finally plasma cells. Differentiation is associated with a number of physical and functional changes in the cells that are well known to those of skill in the art and described herein. When a B cell differentiates into a plasma cell, the resulting cell is said to be “terminally differentiated” because there is no stage beyond the plasma cell stage. However, as used herein, “differentiation” does not require that the resulting cell be terminally defferentiated. For example, in some embodiments, the B cell is differentiated from a pre-B cell to a naïve B cell.

The term “differentiation” refers to the process by which a cell or population of cells acquire new physical and/or functional characteristics that are distinguishable from the characteristics of the cell or population of cells from which it derives. Often differentiation can be broken down into distinct, progessive stages. For example, hematopoeitic stem cells differentiate into pro-B cells, which differentiate into pre-pre-B cells, which differentiate into pre-B cells, which differentiate into immature B cells, which differentiate into naïve B cells, which differentiate into lymphoblasts, which differentiate into memory B cells or plasma cells (memory B cells further differentiate into plasma cells also). Plasma cells are terminally differentiated. By “stimulate B-cell differentiation,” it is meant that a cell or population of cells is caused to enter one of more stages further in the differentiation pathway (e.g., naïve B cell to plasma cell). By “inhibit B-cell differentiation,” it is meant that a cell or population of cells is prevented from entering one of more stages further in the differentiation pathway even though signals may be present that normally cause B cell differentiation.

The term “purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other component normally present in a solution of the same. The terms “isolated” and “purified” do not encompass nucleic acids or polypeptides present in their natural source.

The term “infection” refers to the introduction of nucleic acids into a suitable host cell by use of a virus or viral vector.

The term “transformation” means introducing DNA into a suitable host cell so that the DNA is replicable, either as an extrachromosomal element, or by chromosomal integration.

The term “transfection” refers to the taking up of an expression vector by a suitable host cell, whether or not any coding sequences are in fact expressed.

The term “intermediate fragment” means a nucleic acid between 5 and 1000 bases in length, and preferably between 10 and 40 bp in length.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum. “Secreted” proteins are also intended to include proteins containing non-typical signal sequences (e.g. Interleukin-1 Beta, see Krasney, P. A. and Young, P. R. (1992) Cytokine 4(2): 134-143) and factors released from damaged cells (e.g. Interleukin-1 Receptor Antagonist, see Arend, W. P. et. al. (1998) Annu. Rev. Immunol. 16:27-55)

The term “primordial germ cells (PGCs)” refers to a small population of cells set aside from other cell lineages particularly from the yolk sac, mesenteries, or gonadal ridges during embryogenesis that have the potential to differentiate in to germ cells and other cells. PGCs are the source from which GSCs and ES cells are derived.

The term “stem cells” refers to cells that have the ability to divide for indefinite periods in culture and to give rise to specialized cells.

The term “germ line stem cells (GSCs)” refers to stem cells derived from primordial stem cells that provide a steady and continuous source of germ cells for the production of gametes.

The term “embryonic stem cells (ES)” refers to a cell, which can give rise to many differentiated cell types in an embryo or an adult, including the germ cells.

The PGCs, the GSCs and the ES cells are capable of self-renewal. Thus these cells not only populate the germ line and give rise to a plurality of terminally differentiated cells, which comprise the adult specialized organs, but are able to regenerate themselves.

The term “totipotent” refers to the capability of a cell to differentiate into all of the cell types of an adult organism.

The term “pluripotent” refers to the capability of a cell to differentiate into a number of differentiated cell types that are present in an adult organism. A pluripotent cell is restricted in its differentiation capability in comparison to a totipotent cell.

Each of the above terms is meant to encompasses all that is described for each, unless the context dictates otherwise.

### NUCLEIC ACIDS AND POLYPEPTIDES OF THE INVENTION

Nucleotide and amino acid sequences of the invention are reported below. Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fe portion of an immunoglobulin. For a bivalent form of the protein, such a fusion could be to the Fe portion of an IgG molecule. Other immunoglobulin isotypes may also be used to generate such fusions. For example, a protein-IgM fusion would generate a decavalent form of the protein of the invention or other inhibitors of the invention.

The present invention also provides both full-length and mature forms (for example, without a signal sequence or precursor sequence) of the disclosed proteins. The full-length form of the such proteins is identified in the sequence listing by translation of the nucleotide sequence of each disclosed clone. The mature form of such protein may be obtained by expression of the disclosed full-length polynucleotide in a suitable mammalian cell or other host cell. The sequence of the mature form of the protein is also determinable from the amino acid sequence of the full-length form. Where protein of the present invention is membrane bound, soluble forms of the protein are also provided. In such forms part or all of the regions causing the protein to be membrane bound are deleted so that the protein is fully secreted from the cell in which it is expressed.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Species homologs of the disclosed polynucleotides and proteins are also provided by the present invention. Species homologs may be isolated and identified by making suitable probes or primers from the sequences provided herein and screening a suitable nucleic acid source from the desired species. The invention also encompasses allelic variants of the disclosed polynucleotides or proteins; that is, naturally-occurring alternative forms of the isolated polynucleotide which also encode proteins which are identical, homologous or related to that encoded by the polynucleotides. The compositions of the present invention include isolated polynucleotides, including recombinant DNA molecules, cloned genes or degenerate variants thereof, especially naturally occurring variants such as allelic variants, novel isolated polypeptides, and antibodies that specifically recognize one or more epitopes present on such polypeptides. Species homologs of the disclosed polynucleotides and proteins are also provided by the present invention. Species homologs may be isolated and identified by making suitable probes or primers from the sequences provided herein and screening a suitable nucleic acid source from the desired species. The invention also encompasses allelic variants of the disclosed polynucleotides or proteins; that is, naturally-occurring alternative forms of the isolated polynucleotide which also encode proteins which are identical, homologous or related to that encoded by the polynucleotides.

Nucleic Acids of the Invention

The isolated polynucleotides of the invention include, but are not limited to, a polynucleotide encoding a polypeptide comprising the amino acid sequence of SEQ ID NOS: 1 or 3.

Nucleic acids encoding IL-1Hy1 are useful for producing recombinant IL-1Hy1 polypeptide. Nucleic acids encoding IL-1Hy1 are described in co-owned U.S. Pat. No. 6,294,655, PCT Publication No. WO 99/51744, and PCT Publication No. WO 01/02571 (incorporated herein by reference in their entirety). Typically, one or more cells are transfected with a polynucleotide encoding IL-1Hy1 causing the cells to produce IL-1Hy1. In certain embodiments, the transfected cells are the cells in which differentiation is stimulated by IL-1Hy1 (e.g., B cells). In other embodiments, other cells are transfected and the IL-1Hy1 produced by the cells stimulates differentiation, and/or proliferation, and/or activation of B cells. The recombinant IL-1Hy1 may be collected and purified, but this is not required. For example, recombinant IL-1Hy1-producing cells in vivo may secrete the recombinant protein. This secreted protein can then contact a B cell and stimulate the B cell's differentiation. Thus, in certain embodiments, one or more cells are contacted with IL-1Hy1-encoding polynucleotides in vivo to produce IL-1Hy1 polypeptides which in turn stimulate B cell differentiation in vivo.

The isolated polynucleotides of the invention further include, but are not limited to a polynucleotide comprising the nucleotide sequence of SEQ ID NOS: 1, 2, 4, or 6; a polynucleotide comprising the full length protein coding sequence of SEQ ID NOS: 1, 2, 4, or 6, and; a polynucleotide comprising the nucleotide sequence of the mature protein coding sequence of SEQ ID NOS: 1, 2, 4, or 6. The polynucleotides of the present invention also include, but are not limited to, a polynucleotide that hybridizes to the complement of the nucleotide sequence of SEQ ID NOS: 1, 2, 4, or 6 under stringent hybridization conditions; a polynucleotide which is an allelic variant of any polynucleotide recited above; a polynucleotide which encodes a species homolog of any of the proteins recited above; or a polynucleotide that encodes a polypeptide comprising a specific domain or truncation of the polypeptide of SEQ ID NOS: 1, 2, 4, or 6.

The isolated polynucleotides encoding a polypeptide with IL-1Hy1 activity further include, but are not limited to a polynucleotide comprising the nucleotide sequence of the genomic clone SEQ ID NOS: 7 or 8; a polynucleotide assembled from one or more of the exons of SEQ ID NOS: 7 or 8 (e.g., alternative splicing); a polynucleotide assembled from one or more of the introns of SEQ ID NOS: 7 or 8; a polynucleotide assembled from one or more of the exons of SEQ ID NOS: 7 or 8 and one or more of the introns of SEQ ID NOS: 7 or 8; a polynucleotide comprising the full length protein coding sequence of SEQ ID NOS: 7 or 8; a polynucleotide comprising the nucleotide sequence of the mature protein coding sequence of SEQ ID NOS: 7 or 8.

The polynucleotides encoding a polypeptide with IL-Hy1 activity also include, but are not limited to, a polynucleotide that hybridizes to the complement of the nucleotide sequence of SEQ ID NOS: 7 or 8 under stringent hybridization conditions; a polynucleotide that hybridizes to the complement of any one of the introns or exons of SEQ ID NOS: 7 or 8 under stringent hybridization conditions; a polynucleotide which is an allelic variant of any polynucleotide recited above; a polynucleotide which encodes a species homolog of any of the proteins recited above; or a polynucleotide that encodes a polypeptide comprising a specific domain or truncation of the polypeptide of SEQ ID NOS: 7 or 8.

The polynucleotides encoding a polypeptide with IL-1Hy1 activity still further include, but are not limited to, a polynucleotide comprising the nucleotide sequence of the cDNA insert of clone pIL-1Hy273 deposited with the American Type Culture Collection (ATCC; 10801 University Blvd., Manassas, Va., 20110-2209, U.S.A.); a polynucleotide comprising a nucleotide sequence encoding the amino acid sequence of SEQ ID NO: 1 or 3 which polynucleotide is assembled from the cDNA insert of clone pIL-1Hy273; a polynucleotide comprising the full length protein coding sequence of SEQ ID NO: 1 or 3 which polynucleotide is assembled from the cDNA insert of clone pIL-1Hy273;or, a polynucleotide comprising the nucleotide sequence of the mature protein coding sequence of SEQ ID NO: 1 or 3.

The polynucleotides of the invention additionally include the complement of any of the polynucleotides recited above.

Nucleic acids encoding a polypeptide with IL-Hy1 activity also include nucleotide sequences that are substantially equivalent to the polynucleotides recited above. Such nucleic acids can have at least about 80%, at least 82%, at least 83%, at least 84%, at least 85%, at lease 86%, at least 87%, at least 88%, at least 89%, at least 90%, at least 91%, at least 92%, at lease 93%, at least 94%, at least about 95%, at least 96%, at least 97%, at least 98%, or at least 99% sequence identity to a polynucleotide recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions which can routinely isolate polynucleotides of the desired sequence identities. Furthermore, methods of determining whether nucleic acids enable polypeptides having, IL-1Hy1 activity (e.g. stimulate B cell differention) are provided herein (see examples).

A polynucleotide according to the invention can be joined to any of a variety of other nucleotide sequences by well-established recombinant DNA techniques (see Sambrook J et al. (1989) Molecular Cloning: A Laboratory Manual, Cold Spring Harbor Laboratory, NY). Useful nucleotide sequences for joining to polypeptides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The polynucleotides of the present invention also make possible the development, through, e.g., homologous recombination or knock out strategies, of animals that fail to express functional IL-1Hy1 or that express a variant of IL-1Hy1. Such animals are useful as models for studying the in vivo activities of IL-1Hy1 as well as for studying modulators of IL-1Hy1

In preferred methods to determine biological functions of the polypeptides of the invention in vivo, one or more genes provided by the invention are either over expressed or inactivated in the germ line of animals using homologous recombination [Capecchi, Science 244:1288-1292 (1989)]. Animals in which the gene is over expressed, under the regulatory control of exogenous or endogenous promoter elements, are known as transgenic animals. Animals in which an endogenous gene has been inactivated by homologous recombination are referred to as “knockout” animals. Knockout animals, preferably non-human mammals, can be prepared as described in U.S. Pat. No. 5,557,032, incorporated herein by reference. Transgenic animals are useful to determine the roles polypeptides of the invention play in biological processes, and preferably in disease states. Transgenic animals are useful as model systems to identify compounds that modulate lipid metabolism. Transgenic animals, preferably non-human mammals, are produced using methods as described in U.S. Pat. No. 5,489,743 and PCT Publication No. WO94/28122, incorporated herein by reference.

Transgenic animals can be prepared wherein all or part of a polynucleotides of the invention promoter is either activated or inactivated to alter the level of expression of the polypeptides of the invention. Inactivation can be carried out using homologous recombination methods described above. Activation can be achieved by supplementing or even replacing the homologous promoter to provide for increased protein expression. The homologous promoter can be supplemented by insertion of one or more heterologous enhancer elements known to confer promoter activation in a particular tissue.

Knowledge of IL-1Hy1 DNA sequences allows for modification of cells to permit, or increase, expression of endogenous IL-1Hy1. Cells can be modified (e.g., by homologous recombination) to provide increased IL-1Hy1 expression by replacing, in whole or in part, the naturally occurring IL-1Hy1 promoter with all or part of a heterologous promoter so that the cells express IL-1Hy1 at higher levels. The heterologous promoter is inserted in such a manner that it is operatively linked to IL-1Hy1 encoding sequences. See, for example, PCT International Publication No. WO94/12650, PCT International Publication No. WO92/20808, and PCT International Publication No. WO91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the IL-1Hy1 coding sequence, amplification of the marker DNA by standard selection methods results in co-amplification of the IL-1Hy1 coding sequences in the cells.

The sequences falling within the scope of the present invention are not limited to the specific sequences herein described, but also include allelic variations thereof. Allelic variations can be routinely determined by comparing the sequence provided in SEQ ID NOS: 2, 4, 5 or 6, a representative fragment thereof, or a nucleotide sequence at least 99.9% identical to SEQ ID NOS: 2, 4, 5 or 6, with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another which encodes the same amino acid is expressly contemplated. Any specific sequence disclosed herein can be readily screened for errors by resequencing a particular fragment, such as an ORF, in both directions (i.e., sequence both strands).

The present invention further provides recombinant constructs comprising a nucleic acid having the sequence of SEQ ID NOS: 2, 4, 5 or 6, or a fragment thereof. The recombinant constructs of the present invention comprise a vector, such as a plasmid or viral vector, into which a nucleic acid having the sequence of SEQ ID NOS: 2, 4, 5 or 6 or a fragment thereof is inserted, in a forward or reverse orientation. In the case of a vector comprising one of the ORFs of the present invention, the vector may further comprise regulatory sequences, including for example, a promoter, operably linked to the ORF. For vectors comprising the EMFs and UMFs of the present invention, the vector may further comprise a marker sequence or heterologous ORF operably linked to the EMF or UMF. Large numbers of suitable vectors and promoters are known to those of skill in the art and are commercially available for generating the recombinant constructs of the present invention. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, pSVL (Pharmacia).

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP 1 gene, and a promoter derived from a highly-expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Included within the scope of the nucleic acid sequences of the invention are nucleic acid sequences that hybridize under stringent conditions to a fragment of the DNA sequences of the invention or its complement, which fragment is greater than about 10 bp, preferably 20-50 bp, and even greater than 100 bp. In accordance with the invention, polynucleotide sequences which encode the novel nucleic acids, or functional equivalents thereof, may be used to generate recombinant DNA molecules that direct the expression of that nucleic acid, or a functional equivalent thereof, in appropriate host cells.

Useful nucleic acid sequences further include sequences which encode variants of SEQ ID NO: 2. These amino acid sequence variants may be prepared by methods known in the art by introducing appropriate nucleotide changes into a native or variant polynucleotide. There are two variables in the construction of amino acid sequence variants: the location of the mutation and the nature of the mutation. The amino acid sequence variants of the nucleic acids are preferably constructed by mutating the polynucleotide to give an amino acid sequence that does not occur in nature. These amino acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

In a preferred method, polynucleotides encoding the novel nucleic acids are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). PCR may also be used to create amino acid sequence variants of the novel nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives the desired amino acid variant.

A further technique for generating amino acid variants is the cassette mutagenesis technique described in Wells et al., Gene 34:315 (1985); and other mutagenesis techniques well known in the art, such as, for example, the techniques in Sambrook et al., supra, and Current Protocols in Molecular Biology, Ausubel et al. Due to the inherent degeneracy of the genetic code, other DNA sequences which encode substantially the same or a functionally equivalent amino acid sequence may be used in the practice of the invention for the cloning and expression of these novel nucleic acids. Such DNA sequences include those which are capable of hybridizing to the appropriate novel nucleic acid sequence under stringent conditions.

Polynucleotides of the invention can also be used to induce immune responses. For example, as described in Fan et al., Nat. Biotech. 17:870-872 (1999), incorporated herein by reference, nucleic acid sequences encoding a polypeptide may be used to generate antibodies against the encoded polypeptide following topical administration of naked plasmid DNA or following injection, and preferably intramuscular injection of the DNA. The nucleic acid sequences are preferably inserted in a recombinant expression vector and may be in the form of naked DNA.

Hosts

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell which drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention, can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, Cv-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989), the disclosure of which is hereby incorporated by reference.

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell tines. Mammalian expression vectors will comprise an origin of replication, a suitable promoter and also any necessary ribosome binding sites, polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the mature protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

Polypeptides of the Invention

The isolated polypeptides of the invention include, but are not limited to, a polypeptide comprising the amino acid sequence of SEQ ID NOS: 3 or 5; a full length protein coding sequence of SEQ ID NOS: 3 or 5; a mature protein coding sequence of SEQ ID NOS: 3 or 5, or; a polypeptide encoded by one or more of the exons of SEQ ID NOS: 7 or 8.

The polypeptides of the present invention further include, but are not limited to, a polypeptide comprising the amino acid sequence encoded by the cDNA insert of clone pIL-1Hy273 deposited with the American Type Culture Collection (ATCC; 10801 University Blvd., Manassas, Va., 20110-2209, U.S.A.); a full length protein of SEQ ID NO: 1 or 3 assembled from the amino acid sequence encoded by the cDNA insert of clone pIL-1Hy273; or, a mature protein coding sequence of SEQ ID NO: 1 or 3 assembled from the amino acid sequence encoded by cDNA insert of clone pIL-1Hy273.

Protein compositions of the present invention may further comprise an acceptable carrier, such as a hydrophilic, e.g., pharmaceutically acceptable, carrier.

The invention also relates to methods for producing a polypeptide comprising growing a culture of the cells of the invention in a suitable culture medium, and purifying the protein from the culture. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, and further purified. Preferred embodiments include those in which the protein produced by such process is a full length or mature form of the protein.

The invention further provides a polypeptide including an amino acid sequence that is substantially equivalent to SEQ ID NOS: 1 or 3. Polypeptides according to the invention can have at least about 95%, and more typically at least about 98%, sequence identity to SEQ ID NOS: 1 or 3.

The present invention further provides isolated polypeptides encoded by the nucleic acid fragments of the present invention or by degenerate variants of the nucleic acid fragments of the present invention. By “degenerate variant” is intended nucleotide fragments which differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins. A variety of methodologies known in the art can be utilized to obtain any one of the isolated polypeptides or proteins of the present invention. At the simplest level, the amino acid sequence can be synthesized using commercially available peptide synthesizers. This is particularly useful in producing small peptides and fragments of larger polypeptides. Fragments are useful, for example, in generating antibodies against the native polypeptide. In an alternative method, the polypeptide or protein is purified from bacterial cells which naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook, et al., in Molecular Cloning: A Laboratory Manual; Ausubel et al., Current Protocols in Molecular Biology.

The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention. The purified polypeptides can be used in in vitro binding assays which are well known in the art to identify molecules which bind to the polypeptides. These molecules include but are not limited to, for e.g., small molecules, molecules from combinatorial libraries, antibodies or other proteins. The molecules identified in the binding assay are then tested for antagonist or agonist activity in in vivo tissue culture or animal models that are well known in the art. In brief, the molecules are titrated into a plurality of cell cultures or animals and then tested for either cell/animal death or prolonged survival of the animal/cells.

In addition, the binding molecules may be complexed with toxins, e.g., ricin or cholera, or with other compounds that are toxic to cells. The toxin-binding molecule complex is then targeted to a tumor or other cell by the specificity of the binding molecule for SEQ ID NO: 1 or 3.

The protein of the invention or other inhibitors of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat.RTM. kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention or other inhibitors of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl.RTM. or Cibacrom blue 3GA Sepharose.RTM.; one or more steps involving hydrophobic interaction chromatography using such resins as phenyl ether, butyl ether, or propyl ether; or immunoaffinity chromatography.

Alternatively, the protein of the invention or other inhibitors of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and In Vitrogen, respectively. The protein can also be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The polypeptides of the invention include IL-1Hy1 analogs. This embraces fragments of IL-1Hy1, as well as IL-1Hy1 which comprise one or more amino acids deleted, inserted, or substituted. Also, analogs of IL-1Hy1 embrace fusions of IL-1Hy1 or modifications of IL-1Hy1, wherein the IL-1Hy1 or analog is fused to another moiety or moieties, e.g., targeting moiety or another therapeutic agent. Such analogs may exhibit improved properties such as activity and/or stability. Examples of moieties which may be fused to IL-1Hy1 or an analog include, for example, targeting moieties which provide for the delivery of polypeptide to desired cells, including bone marrow, antibodies to pancreatic cells, antibodies to immune cells such as T-cells, monocytes, dendritic cells, granulocytes, etc., as well as receptor and ligands expressed on pancreatic or immune cells. Other moieties which may be fused to IL-1Hy1 include therapeutic agents which are used for treatment, for example, immunostimulants, immune modulators, and other cytokines.

Compositions Having IL-1HY1 Activity

In certain embodiments, compositions comprising IL-1Hy1 polypeptides are used to stimulate differentiation, and/or proliferation, and/or activation of B cells. IL-1Hy1 polypeptides are described in co-owned U.S. Pat. No. 6,294,655, PCT Publication No. WO 99/51744, and PCT Publication No. WO 01/02571 (incorporated herein by reference in their entirety). Preferably, the IL-1Hy1 polypeptide comprises the amino acid sequence of SEQ ID NO:3. IL-1Hy1 polypeptides include polypeptides comprising amino acid sequences that are variants of SEQ ID NO:3 yet retain the ability to stimulate the differentiation, and/or proliferation, and/or activation of B cells. Such variants comprise at least 80% at least 82%, at least 83%, at least 84, at least 85%, at least 86%, at least 87%, at least 88%, at least 89%, at least 90%, at least 91%, at least 92%, at least 93%, at least 94, at least 95%, at least 96%, at least 97, at least 98%, or at least 99% identity with SEQ ID NO:3. Furthermore, an IL1-Hy1 polypeptide includes polypeptides comprising the amino acid sequence encoded by the cDNA insert of clone pIL-1Hy273 deposited on Mar. 12, 1999, with the American Type Culture Collection (ATCC; 10801 University Blvd., Manassas, Va., 20110-2209, U.S.A.).

Furthermore, IL-1Hy1 polypeptide is meant to include analogs, fragments, and derivatives of the amino acid sequence of SEQ ID NO:3 that retain the ability to stimulate proliferation or differentiation, and/or proliferation, and/or activation of B cells.

The IL-1Hy1 fragments may also be fused to carrier molecules such as immunoglobulins or fragments thereof for many purposes, including increasing half life or the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein of the invention.

Variants also include IL-1Hy1 polypeptides chemically modified by such techniques as ubiquitination, labeling (e.g., with radionuclides or various enzymes), pegylation (derivatization with polyethylene glycol) and insertion or substitution by chemical synthesis of amino acids such as omithine, which do not normally occur in human proteins.

Compositions of the present invention can include IL-1Hy1 analogs. This embraces fragments of IL-1Hy1, as well as IL-1Hy1 which comprise one or more amino acids deleted, inserted, or substituted. Also, analogs of IL-1Hy1 embrace fusions of IL-1Hy1 or modifications of IL-1Hy1, wherein the IL-1Hy1 or analog is fused to another moiety or moieties, e.g., targeting moiety or another therapeutic agent.

The compositions of the present invention may be concurrently administered with a vaccine to improve the efficacy of the vaccine. Concurrent administration includes delivery of IL-1Hy1 at any point or points in time during which an immune response is elicited by an immunogen, such as a vaccine, as would be known in the art. As used herein, enhancement of vaccine efficacy may be manifested by, for example, an improvement in the titer of relevant antibody post-vaccination, a reduction in the number of vaccinations or the amount of vaccine necessary to elicit an immune response (e.g., an antibody response), and/or a reduction in the time period for vaccine efficacy. IL-1Hy1 may be concurrently administered with any immunogen which elicits an immune response, including, but not limited to, vaccines such as varicella vaccine, measles containing vaccines such as the MMR (measles, mumps, rubella) vaccine, DPT (diphtheria, pertussis, tetanus) vaccine, meningitis vaccines, such as Pnu-Immune and Prenar, oral polio vaccine, Salk polio vaccine, hepatitis B vaccine, small pox vaccine and anthrax vaccine. IL-1Hy1 may also be concurrently administered with gene therapy vaccines.

Compositions comprising IL-1Hy1 activity may further comprise an acceptable carrier, such as a hydrophilic, e.g., pharmaceutically acceptable, carrier.

Gene Therapy

In certain methods of the present invention nucleotides encoding a polypeptide having IL1-Hy1 activity are provided to an animal or human in vivo to treat a disease or infection. Thus, the present invention includes methods of gene therapy. Mutations in the IL-1Hy1 gene that result in loss of normal function of the IL-1Hy1 gene product underlie IL-1Hy1-related human disease states. The invention comprehends gene therapy to restore normal IL-1Hy1 activity or to treating those disease states involving IL-1Hy1. Delivery of a functional IL-1Hy1 gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no 6679, pp. 25-30 (1998). For additional reviews of gene therapy technology, see Friedmann, Science, 244: 1275-1281 (1989); Verma, Scientific American: 68-84 (1990); and Miller, Nature, 357: 455-460 (1992).

Introduction of any one of the nucleotides of the present invention or a gene encoding the polypeptides of the present invention can also be accomplished with extrachromosomal substrates (transient expression) or artificial chromosomes (stable expression). Cells may also be cultured ex vivo transfected with a polynucleotide and then re-introduced for therapeutic purposes.

Alternatively, it is contemplated that in other human disease states, preventing the expression of or inhibiting the activity of IL-1Hy1 will be useful in treating the disease states. It is contemplated that antisense therapy or gene therapy could be applied to negatively regulate the expression of IL-1Hy1. Further, the polypeptides of the present invention can be inhibited by the introduction of antisense molecules that hybridize to nucleic acids that encode IL-1Hy1 and by the removal of the IL-1Hy1 gene.

The present invention still further provides cells genetically engineered in vivo to express the polynucleotides of the invention, wherein such polynucleotides are in operative association with a regulatory sequence heterologous to the host cell which drives expression of the polynucleotides in the cell. These methods can be used to increase or decrease the expression of IL-1Hy1.

Knowledge of DNA sequences provided by the invention allows for modification of cells to permit, increase, or decrease, expression of endogenous polypeptide. Cells can be modified (e.g., by homologous recombination) to provide increased polypeptide expression by replacing, in whole or in part, the naturally occurring promoter with all or part of a heterologous promoter so that the cells express the protein at higher levels. The heterologous promoter is inserted in such a manner that it is operatively linked to the desired protein encoding sequences. See, for example, PCT International Publication No. WO 94/12650, PCT International Publication No. WO 92/20808, and PCT International Publication No. WO 91/09955. It is also contemplated that, in addition to heterologous promoter DNA, amplifiable marker DNA (e.g., ada, dhfr, and the multifunctional CAD gene which encodes carbamyl phosphate synthase, aspartate transcarbamylase, and dihydroorotase) and/or intron DNA may be inserted along with the heterologous promoter DNA. If linked to the desired protein coding sequence, amplification of the marker DNA by standard selection methods results in co-amplification of the desired protein coding sequences in the cells.

In another embodiment of the present invention, cells and tissues may be engineered to express an endogenous IL-1Hy1 gene under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting. These sequence include polyadenylation signals, mRNA stability elements, splice sites, leader sequences for enhancing or modifying transport or secretion properties of the protein, or other sequences which alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques which can be used in accordance with this aspect of the invention are more particularly described in U.S. Pat. No. 5,272,071 to Chappel; U.S. Pat. No. 5,578,461 to Sherwin et al.; International Application No. PCT/US92/09627 (WO93/09222) by Selden et al.; and International Application No. PCT/US90/06436 (WO91/06667) by Skoultchi et al., each of which is incorporated by reference herein in its entirety.

Deposit of Clone

The following clone, pIL-1Hy 273 was deposited with the American Type Culture Collection (ATCC) 10801 University Avenue, Manassas, Va., on Mar. 12, 1999 under the terms of the Budapest Treaty. The 2,648 base pair cDNA insert of clone pIL-1Hy 273 is contained in vector pSPORT1 and is flanked by Not1 and Sal1 restriction sites. The clone represent a plasmid clone as described in the Examples set forth below.

| Microorganism/Clone | ATCC Accession No. |
|---|---|
| pIL-1Hy 273 | 203841 |

Cytokine and Cell Proliferation/Differentiation Activity

A protein or other compositions of the present invention may exhibit cytokine, cell proliferation (either inducing or inhibiting) or cell differentiation (either inducing or inhibiting) activity or may induce production of other cytokines in certain cell populations. A polynucleotide of the invention can encode a polypeptide exhibiting such attributes. Many protein factors discovered to date, including all known cytokines, have exhibited activity in one or more factor-dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, MC(preB M+), 2E8, RB5, DA1, 123, T1165, HT2, CTLL2, TF-1, Mo7e and CMK. The activity of a protein of the invention or other inhibitors of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., I. Immunol. 149:3778-3783, 1992; Bowman et al., I. Immunol. 152:1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human interleukin gamma., Schreiber, R. D. In Current Protocols in Immunology. J. E. e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J. E. e.a. Coligan eds. Vol 1 pp. 6.6.1-6.6.5, John Wiley and Sons, Toronto. 1991; Smith et al, Proc. Natl. Aced. Sci. U.S.A. 83:1857-1861, 1986; Measurement of human Interleukin 11—Bennett, F., Giannotti, J., Clark, S. C. and Turner, K. J. In Current Protocols in Immunology. J. E. e.a. Coligan eds. Vol 1 pp. 6.15.1 John Wiley and Sons, Toronto. 1991; Measurement of mouse and human Interleukin 9—Ciarletta, A., Giannotti, J., Clark, S. C. and Turner, K. J. In Current Protocols in Immunology. J. E. e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Methods of Modulating B Cell Differentiation

B lymphocytes develop from pluripotent hematopoietic stem cells throughout life. Development initiates in the liver before birth and in the bone marrow afterward. Hematopoietic stem cells are CD34+ cells that give rise to a common lymphoid progenitor and a myeloid progenitor. The common lymphoid progenitor gives rise to cells of the B-cell and T-cell lineage. Cells of the B cell lineage in the bone marrow express a form of CD45 known as B220. The bone marrow contains cells within the early stages of B-cell differentiation (pro-B cells, pre-pre-B cells, pre-B cells, and immature B cells). Although B-cell differentiation begins in the bone marrow, it continues within the periphery. The immature B cells differentiate into naïve B cells, which subsequently enter the periphery. Naïve cells develop into IgM-secreting lymphoblast cells, followed by memory B cells, and finally antibody-secreting plasma cells.

Administration of IL-1Hy1 to B cells stimulates B-cell differentiatoin. IL-1Hy1 compositions and methods of making and using such are disclosed in co-owned U.S. Pat. No. 6,294,655, PCT Publication No. WO 99/51744, and PCT Publication No. WO 01/02571 (incorporated herein by reference in their entirety). In some embodiments, such as in vitro embodiments, administration can comprise culturing a stem cell, B-cell progenitor, or B cell in the presence of IL-1Hy1 at an appropriate concentration and time to cause cells within the culture to differentiate or to initiate the differentiation process within the cultured cells. In certain embodiments, the cells are differentiated into plasma cells. Because plasma cells secrete large amounts of antibodies and such antibodies often are high affinity antibodies, the methods of the present invention are useful in increasing the yield of antibody producing cells in a B cell culture, thus increasing the production of antibodies by a culture.

The cultured cells may be a cell line or may be cells isolated from an animal or patient. When from an animal or patient, the cells can be isolated from blood, lymph, spleen, or bone marrow. Alternatively, the cells are isolated from a tissue biopsy.

In other embodiments, such as in vivo embodiments, administration comprises contacting B cells or B-cell precursors within an animal or human patient with a composition comprising IL-1Hy1. Because certain diseases are the result of aberrant B cell development, the methods of the present invention include therapeutic methods for treating B cell development-related diseases, such as immunoglobulin deficiency syndromes. In other embodiments, the therapeutic methods of the present invention include methods of treating infection.

The present invention also includes methods of preventing or inhibiting the differentiation of B cells. In such methods, administration of an IL-1Hy1 inhibitor to B cells prevents the B cells from differentiating further. Such methods are useful in treating diseases or disorders wherein inhibition of B cell development is desirable, e.g., multiple myelomas or autoimmune diseases that result in autoantibody production, such as systemic lupus erythmatosus, Crohn's disease, graft-versus-host disease, and asthma.

In certain instances it may be necessary to evaluate a B cell or population containing B cells to determine in which stage of B cell differentiation the cell is. Although some stages of B cell differentiation can be determined by the morphological features of the cells, it is preferred that the stage of development is determined by expression of certain markers. Markers for determining B cell differentiation are well known in the art. The markers include certain nucleic acids or protein expressed during on or more stages of B cell differentiation. In preferred embodiments, the marker is a surface protein or proteins and is detected using antibodies to the protein.

Examples of markers for detecting plasma cells include CD38(human), CD138(mouse and human), XBP-1 (Reimold et al., Nature 412(6844):300-307, 2001), and Blimp-1 (Angelin-Duclos, J. Immunol. 165 (10):5462-71, 2000).

Of course, determination of the lack of a marker on or in a cell can be used (e.g., CD19 is not expressed on plasma cells). Furthermore, detection of combinations of markers may be used to determine differentiation, and/or proliferation, and/or activation of B cells. For example, detection of plasma cell may be determined by detection of CD138, XBP-1, and the lack of detection of CD19.

Immune Stimulating or Suppressing Activity

An IL-1Hy1 protein of the present invention may exhibit immune stimulating activity, including without limitation the activities for which assays are described herein. Thus, inhibitors of the invention may suppress undesirable immune activity. A polynucleotide of the invention can encode a polypeptide exhibiting such activities. A protein may be useful in the treatment of various immune deficiencies and disorders (including severe combined immunodeficiency (SCID)), e.g., in promoting growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpes viruses, mycobacteria, Leishmania spp., malaria spp. and various fungal infections such as candidiasis. IL-1Hy1 stimulates B cell proliferation and B cell activity. Therefore, IL-1Hy1 may effectively treat infection and disorders related to B cell deficiencies. Of course, in this regard, proteins of the present invention may also be useful where a boost to the immune system generally may be desirable, i.e., in the treatment of cancer.

IL-1 has been indicated to promote tumor cell growth in cancers of various organs including breast adenocarcinoma, brain tumors, melanoma, mycloma, giant cell tumors of bone, acute myelogenous leukemia, oral epidermoid carcinoma and squamous cell carcinoma. Similarly, IL-1Hy1 stimulates B cell proliferation. Thus, treatment of B cell related cancer disease states with inhibitors of IL-1Hy1 activity is expected to ameliorate signs and symptoms of these cancers.

IL-1Hy1 inhibits production of IgA. Therefore, autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein (or antagonists thereof, including antibodies) of the present invention may also to be useful in the treatment of allergic reactions and conditions (e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis and contact allergies), such as asthma (particularly allergic asthma) or bronchitis (including chronic bronchitis) and other respiratory problems.

Other conditions, in which immune suppression is desired (including, for example, organ transplantation), may also be treatable using a protein (or antagonists thereof) of the present invention. The therapeutic effects of IL-1Hy1 polypeptides or antagonists thereof on allergic reactions can be evaluated by in vivo animals models such as the cumulative contact enhancement test (Lastbom et al., Toxicology 125: 59-66, 1998), skin prick test (Hoffmann et al., Allergy 54: 446-54, 1999), guinea pig skin sensitization test (Vohr et al., Arch. Toxocol. 73: 501-9), and murine local lymph node assay (Kimber et al., J. Toxicol. Environ. Health 53: 563-79).

Using the proteins of the invention it may also be possible to modulate immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a therapeutic composition of the invention may prevent cytokine synthesis by immune cells, such as T cells, and thus acts as an immunosuppressant. Moreover, a lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular therapeutic compositions in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of therapeutic compositions of the invention on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block stimulation of T cells can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (e.g., a B lymphocyte antigen function), as a means of up regulating immune responses, may also be useful in therapy. Upregulation of immune responses may be in the form of enhancing an existing immune response or eliciting an initial immune response. For example, enhancing an immune response may be useful in cases of viral infection, including systemic viral diseases such as influenza, the common cold, and encephalitis.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

A peptide of the present invention may provide the necessary stimulation signal to T cells to induce a T cell mediated immune response against the transfected tumor cells. In addition, tumor cells which lack MIIC class I or MHC class II molecules, or which fail to reexpress sufficient mounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention or other inhibitors of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al, J. Immunol. 135:1564-1572, 1985; Takai et al., I. Immunol. 137:3494-3500, 1986; Takai et al., J. Immunol. 140:508-512, 1988; Herrmann et al, Proc. Natl. Acad. Sci. USA 78:2488-2492, 1981; Herrmann et al, J. Immunol. 128:1968-1974, 1982; Handa et al., J. Immunol. 135:1564-1572, 1985; Takai et al., J. Immunol. 137:3494-3500, 1986; Bowman et al, J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al, J. Immunol. 149:3778-3783, 1992.

Dendritic cell-dependent assays (which will identify, among others, proteins expressed by dendritic cells that activate naive T-cells) include, without limitation, those described in: Guery et al, J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad. Sci. USA 88:7548-7551, 1991.

Hematopoiesis Regulating Activity

A protein or composition of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in vivo or ex vivo (i.e., in conjunction with bone marrow transplantation or with peripheral progenitor cell transplantation (homologous or heterologous)) as normal cells or genetically manipulated for gene therapy.

The activity of a protein of the invention or other inhibitors of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoictic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. A polynucleotide of the invention can encode a polypeptide exhibiting such attributes. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention or other inhibitors of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Marguiles, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al. Eur. J. Immunol. 25:1744-1748; Gruber et al. J. of Immunol. 152:5860-5867, 1994; Johnston et al. J. of Immunol. 153:1762-1768, 1994.

Receptor/Ligand Activity

A protein of the present invention may also demonstrate activity as receptors, receptor ligands or inhibitors or agonists of receptor/ligand interactions. A polynucleotide of the invention can encode a polypeptide exhibiting such characteristics. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention or other inhibitors of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

By way of example, the novel IL-1Hy1 may be used as a ligand for a cytokine receptor thereby modulating the biological activity of that receptor. Examples of cytokine receptors that may be used include, but are not limited to, the Interleukin-1 Type I or the Interleukin-Type II Receptor and other interleukin receptors. Whether the novel IL-1Hy1 exhibits agonist, partial agonist, antagonist, or partial antagonist activity for a particular receptor, such as a cytokine receptor, in a particular cell type can be determined by conventional techniques known to those skilled in the art. In one embodiment, one or more cells expressing a cytokine receptor (e.g., Interleukin-1 Type I or Type II Receptors) are contacted with the protein of the invention or other inhibitors of the invention. Examples of cells that may be contacted with the protein of the invention or other inhibitors of the invention include, but are not limited to, mammalian cells such as fibroblasts and B cells.

Studies characterizing drugs or proteins as agonist or antagonist or partial agonists a partial antagonist require the use of other proteins as competing ligands. The novel protein of the present invention exhibit an affinity for Interleukin-1 Receptor. Thus, the proteins of the present invention may be used, for example, as competitors in assays involving Interleukin-1 Receptors. Alternatively, the protein of the invention or other inhibitors of the invention may be labeled by being coupled to radioisotopes, calorimetric molecules or a toxin molecules by conventional methods. (“Guide to Protein Purification” Murray P. Deutscher (ed) Methods in Enzymology Vol. 182 (1990) Academic Press, Inc. San Diego) and used in both in vivo and in vitro to bind to the Interleukin-1 Receptor. Examples of radioisotopes include, but are not limited to, tritium and carbon-14. Examples of colorimetric molecules include, but are not limited to, fluorescent molecules such as fluorescamine, or rhodamine or other calorimetric molecules. Examples of toxins include, but are not limited, to riacin. By way of example, the proteins coupled to such molecules are useful in studies involving in vivo or in vitro metabolism of the Interleukin-1 Receptor.

Drug Screening with the Novel IL-1HY1 Polypeptides.

This invention is particularly useful for screening compounds by using the novel IL-1Hy1 polypeptides or binding fragments thereof in any of a variety of drug screening techniques. The novel IL-1Hy1 polypeptides or fragments employed in such a test may either be free in solution, affixed to a solid support, borne on a cell surface or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. Such cells, either in viable or fixed form, can be used for standard binding assays. One may measure, for example, the formation of complexes between novel IL-1Hy1 polypeptides or fragments and the agent being tested or examine the diminution in complex formation between the novel IL-1Hy1 polypeptides and an appropriate cell line, which are well known in the art.

B Cell Related Disorders

Mature B cells are derived from the bone marrow precursor cells and make up about 10-15% of the peripheral blood lymphocytes, 50% of the splenic lymphocytes and about 10% of the bone marrow lymphocytes. The primary function of the B cells is to produce antibodies. B cell development, differentiation and proliferation is regulated by cytokines. In particular, it is known that IL-7 drives pro-B and pre-pre B cell proliferation and differentiation. BCG-F (low-molecular weight B cell growth factor) and IL-1 induce B cell precursor proliferation. IL-1, IL-2, IL-4, IL-5 and IL-6 are known induce mature B cell proliferation and to drive differentiation into antibody secreting cells. In the Examples, it is demonstrated that B cell differentiation is stimulated by IL-1Hy1.

Furthermore, it is demonstrated that IL-1Hy1 stimulates mature B cell proliferation. IL-1Hy1 may induce B cell proliferation by activating intracellular signaling pathways. In Examples 8 and 9, IL-1Hy1 increased total tyrosine phosphorylation of proteins in B cells. In particular, IL-1Hy1 activates the transcription factors ERK, STAT3, p38 and JNK, as indicated by increased phosphoylation.

Leukemias can result from uncontrolled B cell proliferation initially within the bone marrow before disseminating to the peripheral blood, spleen, lymph nodes and finally to other tissues. Uncontrolled B cell proliferation also may result in the development of lymphomas that arise within the lymph nodes and then spread to the blood and bone marrow. Altering the differentiation, and/or proliferation, and/or activation of B cells, thereby preventing the production of B cells of a certain stage of development or facilitating the development of a B cell to a later stage of development, or inhibiting B cell proliferation may be effective in treating B cell malignancies, leukemias, lymphomas and myelomas including but not limited to multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma (NHL), B cell chronic lymphocytic leukemia (CLL), acute lymphoblastic leukemia, hairy cell leukemia (HCL), splenic marginal zone lymphoma, large B cell lymphoma, prolymphocytic leukemia (PLL), lymphoplasma cytoid lymphoma, mantle cell lymphoma, mucosa-associated lymphoid tissue (MALT) lymphoma, primary thyroid lymphoma, intravascular malignant lymphomatosis, splenic lymphoma, and intragraft angiotropic large-cell lymphoma. Other diseases that may be treated by the methods of the present invention include multicentric Castleman's disease, primary amyloidosis, Franklin's disease, Seligmann's disease, primary effusion lymphoma, post-transplant lymphoproliferative disease (PTLD) [associated with EBV infection.], paraneoplastic pemphigus, chronic lymphoproliferative disorders, X-linked lymphoproliferative syndrome (XLP), acquired angioedema, angioimmunoblastic lymphadenopathy with dysproteinemia, Herman's syndrome, post-spenectomy syndrome, congenital dyserythropoietic anemia type III, lymphoma-associated hemophagocytic syndrome (LAHS), necrotizing ulcerative stomatitis, Kikuchi's disease, lymphomatoid granulomatosis, Richter's syndrome, polycythemic vera (PV), Gaucher's disease, Gougerot-Sjogren syndrome, Kaposi's sarcoma, cerebral lymphoplasmocytic proliferation (Bind and Neel syndrome), diseases of the lips, lymphoplasma cellular disorders, post-transplantational plasma cell dyscrasias, and Good's syndrome.

Autoimmune diseases can be associated with hyperactive B cell activity which results in autoantibody production. Inhibition of the development of autoantibody producing cells or proliferation of such cells may be therapeutically effective in decreasing the levels of autoantibodies in autoimmune diseases including but not limited to systemic lupus erythematosus, Crohn's Disease, graft-verses-host disease, Graves' disease, myasthenia gravis, autoimmune hemolytic anemia, autoimmune thrombocytopenia, asthma, cryoglubulinemia, primary biliary schlerosis, pernicious anemia, Waldenstrom macroglobulinemia, hyperviscosity syndrome, macroglobulinemia, cold agglutinin disease, monoclonal gammopathy of undetermined origin, anetoderma, POEMS syndrome (polyneuropathy, organomegaly, endocrinopathy, M component, skin changes), connective tissue disease, multiple sclerosis, rheumatoid arthritis, autoimmune pulmonary inflammation, psoriasis, Guillain-Barre syndrome, autoimmune thyroiditis, insulin dependent diabetes mellitis, autoimmune inflammatory eye disease, Goodpasture's disease, Rasmussen's encephalitis, dermatitis herpetiformis, thyoma, autoimmune polyglandular syndrome type 1, primary and secondary membranous nephropathy, cancer-associated retinopathy, autoimmune hepatitis type 1, mixed cryoglobulinemia with renal involvement, cystoid macular edema, endometriosis, IgM polyneuropathy (including Hyper IgM syndrome), demyelinating diseases, angiomatosis, and monoclonal gammopathy. The autoimmune diseases contemplated by the invention may optionally include those not directly and/or primarily a result of IL-1 β, 1β, I1-12 and/or IL-18 induced inflammation.

Inhibition of IL-1Hy1 activity (or antagonists thereof, including antibodies) may also to be useful in the treatment of allergic reactions and conditions e.g., anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, allergic gastroentreropathy, inflammatory bowel disorder (IBD), contact allergies, such as asthma (particularly allergic asthma) or other respiratory problems.

The stimulation of B cell proliferation may be desired to treat immune deficiency disorders which are associated with reduced levels of circulating antibodies or other conditions, such as infection, wherein increased B cell activity is desirable. Administration of IL-1Hy1 to stimulate differentiation and/or proliferation and/or activation of B cell populations would be useful for the treatment of immune deficiency disorders including but not limited to severe combined immunodefiency syndrome (SCID), adenosine deaminase (ADA) deficiency, purine nucleoside phosphorylase (PNP) deficiency, MHC class II deficiency, immunodefeciency with thymona, reticular dysgenesis and Omenn syndrome (OS). Administration of IL-1Hy1 to stimulate B cell proliferation and differentiation would also be useful in treating immunoglobulin deficiency syndromes including but not limited to agammagglobulinemia, transient hypogammaglobulinemia of infancy, isolated deficiency of IgG, common variable immunodeficiency, X-linked immunodeficiency with increased levels of IgM, isolated deficiency of IgM and linear IgA dermatosis.

Furthermore, administration of IL-1Hy1 to stimulate differentiation and/or proliferation and/or activation of B cell populations would be useful for the treatment of infectious diseases including viral, bacterial, fungal, protozoan and parasitic infections. Examples of viral infections that may be treated include infections caused by Ebstein Barr virus (EBV), cytomegalovirus (CMV), human immunodeficiency virus (HIV), Herpes Simplex virus (HSV), influenza virus, human papilloma virus (HPV), polio, and hepatitis C. Examples of bacterial infections that may be treated include those caused by Bacillus, Bacteroides, Borrelia, Cardiobacterium, Chlamydia, Clostridium, Coxiella, Cryptobacterium, Enterobacter, Haemophilus, Helicobacter, Klebsiella, Legionella, Listeria, Mycobacterium, Neisseria, Pseudomonas, Rickettsia, Salmonella, Serratia, Shigella, Staphylococcus, Streptococcus, Treponema, Vibrio, and Yersinia. Examples of fungal infections that may be treated include those caused by Candida, Trichophyton, Epidermophyton, and Microsporum. Examples of protozoan infections that may be treated include those caused by Cyclospora, Leishmania, Giardia, Cryptosporidia, and Trypanosoma. Examples of parasitic infections that may be treated include hydatid disease, Neurocysticercosis, sleeping sickness, Toxoplasmosis, and diseases caused by tapeworm, roundworm, flukes, and amoebae.

As described briefly above, B cell related disorders may either have increased levels of B cells (e.g. leukemias), hyperactivated B cells (e.g. autoimmune diseases) or decreased levels of B cells (e.g. immune deficiency syndromes). As described in the Examples, IL-1Hy1 may be administered to simulate B cell proliferation or differentiation. Therefore, IL-1Hy1 may be administered to stimulate B cell differentiation or proliferation in immune deficiency syndromes while inhibition of IL-1Hy1 activity may be useful in treatment of B cell disorders such as autoimmune diseases. The B cell related disorders contemplated by the present invention optionally may include those not directly and/or primarily a result of IL-1β, IL-12 and/or IL-18 induced inflammation.

To treat B cell-related disorders, IL-1Hy1 polypeptide or an inhibitor of IL-1Hy1 activity are administered to a patient suffering from said disorder in an effective amount to either stimulate or inhibit B cell differentiation. B cell differentiation can be measured by quantitating the level of B cells at one or more stages of B cell development within a fluid or tissue sample of the treated patient including fluid samples such as blood, plasma, serum, lymphatic fluid samples and tissue samples including bone marrow and spleen samples. B cell differentiation can also be measured indirectly by measuring the level of antibodies within in a fluid or tissue sample of the treated patient.

In one embodiment, the invention contemplates treatment of one or more disease not disclosed in U.S. Pat. No. 6,294,655, PCT Publication No. WO 99/51744, or PCT Publication No. WO 01/02571.

Leukemias and Lymphomas

Leukemias, lymphomas and related disorders may be treated or prevented by administration of a therapeutic that inhibits function of IL-1Hy1 polynucleotides and/or polypeptides. Such leukemias, lymphomas and related disorders include but are not limited to acute leukemia, acute lymphocytic leukemia, acute myelocytic leukemia, myeloblastic, promyelocytic, myelomonocytic, monocytic, erythroleukemia, hairy cell leukemia, chronic leukemia, chronic myelocytic (granulocytic) leukemia, chronic lymphocytic leukemia, multiple myeloma, Burkitt's lymphoma, cutaneous B cell lymphoma, primary follicular cutaneous B cell lymphoma, B lineage acute lymphoblastic leukemia (ALL), B cell non-Hodgkin's lymphoma.(for a review of such disorders, see Fishman et al., 1985, Medicine, 2d Ed., J.B. Lippincott Co., Philadelphia).

Therapeutic Methods

The proteins, polynucleotides and inhibitors of the invention have numerous applications in a variety of therapeutic methods. Examples of therapeutic applications include, but are not limited to, those exemplified below.

Arthritis and Inflammation

The therapeutic effects of inhibiting IL-1Hy1 activity to treat rheumatoid arthritis is determined in an experimental animal model system. The experimental model system is adjuvant induced arthritis in rats, and the protocol is described by J. Holoshitz, et al., 1983, Science, 219:56, or by B. Waksman et al., 1963, Int. Arch. Allergy Appl. Immunol., 23:129. Induction of the disease can be caused by a single injection, generally intradermally, of a suspension of killed Mycobacterium tuberculosis in complete Freund's adjuvant (CFA). The route of injection can vary, but rats may be injected at the base of the tail with an adjuvant mixture. The inhibitor is administered in phosphate buffered solution (PBS) at a dose of about 1-5 mg/kg. The control consists of administering PBS only.

The procedure for testing the effects of an IL-1Hy1 inhibitor would consist of intradermally injecting killed Mycobacterium tuberculosis in CFA followed by immediately administering the inhibitor and subsequent treatment every other day until day 24. At 14, 15, 18, 20, 22, and 24 days after injection of Mycobacterium CFA, an overall arthritis score may be obtained as described by J. Holoskitz above. An analysis of the data would reveal that the inhibitor would have a dramatic affect on the swelling of the joints as measured by a decrease of the arthritis score.

Diabetes

Interleukin-1 has been shown to be involved in the destruction of islet cells in diabetes mellitus (DM) (Mandrup-Paulsen, T., K. Bendtzen, J. Nerup, C. A. Dinarello, M. Svenson, and J. H. Nielson [1986] Diabetologia 29:63-67). Therefore, IL-1Hy1 may play a role in diabetes progression also. Inhibition of IL-1Hy1 activity may limit lymphocyte and macrophage mediated damage to islet cells in incipient cases of DM identified by disease susceptibility via genetic background and family history. The inflammatory destruction of the pancreatic beta islet cells in such individuals with early DM may be reduced by parenterally administering an IL-1Hy1 inhibitor which has an anti-IL-1Hy1 effect in the pancreas.

Cystic Fibrosis

The Examples provided herein demonstrate elevated levels of IL-1Hy1-expressing plasma cells in lungs of cystic fibrosis patients as compared to the lungs of normal individuals. Therefore, IL-1Hy1 may play a role in the development or exacerbation of the diseased state. Inhibition of IL-1Hy1 activity may limit lymphocyte and macrophage mediated damage lungs of cystic fibrosis patients or patients identified as susceptible to cystic fibrosis via genetic background. The inflammatory destruction of the lung tissue in such individuals with early cystic fibrosis may be reduced by administering an IL-1Hy1 inhibitor to the lungs of the patient.

Pharmaceutical Formulations and Routes of Administration

A protein or inhibitor of the present invention (from whatever source derived, including without limitation from recombinant and non-recombinant sources) may be administered to a patient in need, by itself, or in pharmaceutical compositions where it is mixed with suitable carriers or excipient(s) at doses to treat or ameliorate a variety of disorders. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, G-CSF, Meg-CSF, thrombopoietin, stem cell factor, and erythropoietin.

The pharmaceutical composition may further contain other agents which either enhance the activity of the protein or complement its activity or use in treatment. Such additional factors and/or agents may be included in the pharmaceutical composition to produce a synergistic effect with protein of the invention or other inhibitors of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. A protein of the present invention may be active in multimers (e.g., heterodimers or homodimers) or complexes with itself or other proteins. As a result, pharmaceutical compositions of the invention may comprise a protein of the invention or other inhibitors of the invention in such multimeric or complexed form.

Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other inhibitor of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

Alternately, one may administer the compound in a local rather than systemic manner, for example, via injection of the compound directly into a arthritic joints or in fibrotic tissue, often in a depot or sustained release formulation. In order to prevent the scarring process frequently occurring as complication of glaucoma surgery, the compounds may be administered topically, for example, as eye drops. Furthermore, one may administer the drug in a targeted drug delivery system, for example, in a liposome coated with a specific antibody, targeting, for example, arthritic or fibrotic tissue. The liposomes will be targeted to and taken up selectively by the afflicted tissue.

Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the compounds of the invention may be provided as salts with pharmaceutically compatible counterions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the pharmaceutical composition of the invention. The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The amount of protein or other inhibitor of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein of the present invention per kg body weight. For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention or other inhibitors of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells. In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention. The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein. For any compound used in the method of the invention, the therapeutically effective dose can be estimated initially from cell culture assays. For example, a dose can be formulated in animal models to achieve a circulating concentration range that includes the IC₅₀ as determined in cell culture. Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1. Dosage amount and interval may be adjusted individually to provide plasma levels of the active moiety which are sufficient to maintain the desired therapeutic effects, or minimal effective concentration (MEC). The MEC will vary for each compound but can be estimated from in vitro data; for example, the concentration necessary to achieve 50-90% inhibition of cytokine activity using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Antibodies

Also included in the invention are antibodies to proteins, or fragments of proteins of the invention. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, Fab, Fab′ and F(ab′)2 fragments, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species.

An isolated related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence shown in SEQ ID NO: 257-512, or 676-838, or Tables 3, 5, 6, or 8, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a surface region of the protein, e.g., a hydrophilic region. A hydrophobicity analysis of the human related protein sequence will indicate which regions of a related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

The term “specific for” indicates that the variable regions of the antibodies of the invention recognize and bind polypeptides of the invention exclusively (i.e., able to distinguish the polypeptide of the invention from other similar polypeptides despite sequence identity, homology, or similarity found in the family of polypeptides), but may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the polypeptides of the invention are also contemplated, provided that the antibodies are first and foremost specific for, as defined above, full-length polypeptides of the invention. As with antibodies that are specific for full length polypeptides of the invention, antibodies of the invention that recognize fragments are those which can distinguish polypeptides from the same family of polypeptides despite inherent sequence identity, homology, or similarity found in the family of proteins.

Antibodies of the invention are useful for, for example, therapeutic purposes (by modulating activity of a polypeptide of the invention), diagnostic purposes to detect or quantitate a polypeptide of the invention, as well as purification of a polypeptide of the invention. Kits comprising an antibody of the invention for any of the purposes described herein are also comprehended. In general, a kit of the invention also includes a control antigen for which the antibody is immunospecific. The invention further provides a hybridoma that produces an antibody according to the invention. Antibodies of the invention are useful for detection and/or purification of the polypeptides of the invention.

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and Sepharose®, acrylic resins and such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Computer Readable Sequences and Structural Coordinates

According to one aspect of this invention, a nucleotide sequence, amino acid sequence or three-dimensional structure of the present invention can be recorded on computer readable media. A three-dimensional structure may be represented or displayed using structural coordinates of atoms of amino acids within amino acid sequences of the present invention (including mutant or variant amino acid sequences), particularly amino acids involved in binding to IL-1 receptor or other receptors or IL-1 receptor accessory protein, as well as amino acids involved in other IL-1Hy1 functions.

As used herein, “computer readable media” or “machine readable storage medium” refers to any medium which can be read and accessed directly by a computer. The term “data storage material” refers to any material on which data can be physically stored in. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The term “machine readable data” refers to a group of one or more characters, including numbers, representing basic elements of information that can be processed by a computer. A skilled artisan can readily appreciate how any of the presently known computer readable media can be used to create a manufacture comprising a computer readable medium having recorded thereon a nucleotide sequence, amino acid sequence or structural coordinates of the present invention that can be used to render a three-dimensional structure of a polypeptide.

As used herein, “recorded” refers to a process for storing information on computer readable medium. A skilled artisan can readily adopt any of the presently known methods for recording information on computer readable medium to generate manufactures comprising the sequence or structure information of the present invention. A variety of data storage structures are available to a skilled artisan for creating a computer readable medium having recorded thereon sequence or structure information of the present invention. The choice of the data storage structure will generally be based on the means chosen to access the stored information. In addition, a variety of data processor programs and formats can be used to store the sequence or structure information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of dataprocessor structuring formats (e.g. text file or database) in order to obtain computer readable medium having recorded thereon the sequence or structure information of the present invention.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the present invention. As stated above, the computer-based systems of the present invention comprise a data storage means having stored therein sequence or structure information of the present invention and the necessary hardware means and software means for supporting and implementing a search means. As used herein, “data storage means” refers to memory which can store sequence or structure information of the present invention, or a memory access means which can access manufactures having recorded thereon the sequence or structure information of the present invention.

Input means can be implemented in a variety of ways. Machine-readable data of this invention may be inputted via the use of a modem or modems connected by a telephone line or dedicated data line. Alternatively or additionally, the input means may comprise CD-ROM drives or disk drives. In conjunction with a display terminal, a keyboard may also be used as an input device. Output means may similarly be implemented by conventional devices. By way of example, output hardware may include CRT display terminal for displaying a graphical representation of important functional residues of the invention using a computer program as described herein. Output means might also include a printer, so that hard copy output may be produced, or a disk drive to store system output for later use.

In operation, the CPU coordinates the use of the various input and output devices, coordinates data accesses from data storage means including working memory, and determines the sequence of data processing steps. A number of programs may be used to process the machine-readable data of the invention, to form or display a sequence or a three-dimensional structure or representation, or to carry out computational methods of sequence comparison or drug discovery.

For example, by providing the nucleotide sequence of SEQ ID NOS: 2, 4 or 5 or a representative fragment thereof, or a nucleotide sequence at least 99.9% identical to SEQ ID NOS: 2, 4 or 5 or 6 in computer readable form, a skilled artisan can routinely access the sequence information for a variety of purposes. Computer software is publicly available which allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system is used to identify open reading frames (ORFs) within a nucleic acid sequence. Such ORFs may be protein encoding fragments and may be useful in producing commercially important proteins such as enzymes used in fermentation reactions and in the production of commercially useful metabolites.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computational methods of drug discovery may include computational evaluation of a three-dimensional structure for its ability to associate with moieties of chemical compounds. This evaluation may include performing a fitting operation between the structure or a portion thereof and one or more moieties of a chemical compound, and thereby qualitatively or quantitatively judging the proximity and/or extent of interaction between the three-dimensional structure and the chemical moiety(ies). Interaction may take place through, e.g., non-covalent interactions such as hydrogen bonding, van der Waals interactions, hydrophobic interactions and electrostatic interactions, or through covalent bonding. When the structure is displayed in a graphical three-dimensional representation on a computer screen, this allows visual inspection of the structure, as well as visual inspection of the structure's association with chemical moieties.

Specialized computer programs may be used to assist in a process of selecting chemical moieties or fragments of chemical compounds for further evaluation. These include: 1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK. 2. MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif. 3. AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif 4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Assembly of individual chemical moieties or fragments can be assisted by using programs including: 1. CAVEAT (P. A. Bartlett et al., “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des., 8, pp.51-66 (1994)). CAVEAT is available from the University of California, Berkeley, Calif. 2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992). 3. HOOK (M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Computer programs that assist in designing a chemical compound that potentially interacts with a three-dimensional structure as a whole or “de novo” using either an empty binding site or optionally including some portion(s) of a known modulator(s) include: 1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif. 2. LEGEND (Y. Nishibata et al., Tetrahedron, 47, p. 8985 (1991)). LEGEND is available from Molecular Simulations Incorporated, San Diego, Calif. 3. LeapFrog (available from Tripos Associates, St. Louis, Mo.). 4. SPROUT (V. Gillet et al., “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., N.C. Cohen et al., “Molecular Modeling Software and Methods for Medicinal Chemistry”, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modem Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Binding affinity may be tested and optimized by computational evaluation, e.g. by minimizing the energy between the bound and free states of the three-dimensional structure (e.g., a small deformation energy of binding, preferably not greater than about 10 kcal/mole and more preferably not greater than 7 kcal/mole).

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs may be implemented, for instance, using a Silicon Graphics workstation with “IMPACT” graphics. Other hardware systems and software packages will be known to those skilled in the art.

Such computational drug design may include computer-based screening of small molecule databases for chemical moieties or chemical compounds that can bind in whole, or in part, to the desired three-dimensional structure. In this screening, the quality of fit of such entities to the binding site may be judged either by shape complementarity or by estimated interaction energy [E. C. Meng et al., J. Comp. Chem., 13, pp. 505-524 (1992)].

Triple Helix Formation

In addition, the fragments of the present invention, as broadly described, can be used to control gene expression through triple helix formation or antisense DNA or RNA, both of which methods are based on the binding of a polynucleotide sequence to DNA or RNA. Polynucleotides suitable for use in these methods are usually 20 to 40 bases in length and are designed to be complementary to a region of the gene involved in transcription (triple helix—see Lee et al., Nucl. Acids Res. 6:3073 (1979); Cooney et al., Science 15241:456 (1988); and Dervan et al., Science 251:1360 (1991)) or to the mRNA itself (antisense—Olmno, J. Neurochem. 56:560 (1991); Oligodeoxynucleotides as Antisense Inhibitors of Gene Expression, CRC Press, Boca Raton, Fla. (1988)). Triple helix—formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide.

Diagnostic Assays and Kits

The present invention further provides methods to identify the presence or expression of one of the ORFs of the present invention, or homolog thereof, in a test sample, using a nucleic acid probe or antibodies of the present invention.

In general, methods for detecting a polynucleotide of the invention can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

Screening Assays

The present invention further provides methods of obtaining and identifying agents that alter B cell differentiation. In a preferred embodiment, the method comprises the steps of:

(a) incubating a population of cells containing one or more naïve B cells with an effective amount of IL-1Hy1 in the presence and absence of a test compound;

(b) determining the effect of the test compound on the ability of IL-1Hy1 to stimulate B cell differentiation.

The results obtained using the test compound may be compared to controls. The controls may be compounds known to prevent the ability of IL-1Hy1 to stimulate B cell differentiation, compounds known not known to prevent the ability of IL-1Hy1 to stimulate B cell differentiation; or the control may be the absence of a compound.

The test compound may be a small molecule or a polypeptide such as an IL-1Hy1 variant or mutant, soluble receptor, or an antibody. The test compounds can be, but are not limited to, peptides, carbohydrates, vitamin derivatives, or other pharmaceutical agents. The agents can be selected and screened at random or rationally selected or designed using protein modeling techniques.

Because compounds that bind IL-Hy1 can inhibit the protein's function, it may be beneficial to screen for compounds that bind IL-Hy1. Screening for such compounds may include the steps of:

(a) contacting a test compound with an isolated IL-Hy1 protein; and

(b) determining whether the test compound binds to said protein.

In general, therefore, such methods for identifying compounds that bind to IL-Hy1 can comprise contacting a compound with IL-Hy1 for a time sufficient to form an IL-Hy1/compound complex, and detecting the complex, so that if an IL-Hy1/compound complex is detected, a compound that binds to IL-Hy1 is identified.

Compounds identified via such methods can include compounds, which modulate the activity of IL-Hy1 (that is, increase or decrease its activity, relative to activity observed in the absence of the compound). Alternatively, compounds identified can include compounds, which modulate the expression of IL-Hy1 polypeptide (that is, increase or decrease expression relative to expression levels observed in the absence of the compound). Compounds, such as compounds identified via the methods of the invention, can be tested using standard assays well known to those of skill in the art for their ability to modulate activity/expression. Also computer based drug design described below can be used to identify modulatory compounds. Computer based drug design to find binders of and modulators of IL-Hy1 is described in co-owned U.S. Pat. No. 6,294,655 (incorporated herein by reference).

For random screening, agents such as peptides, carbohydrates, pharmaceutical agents and the like are selected at random and are assayed for their ability to bind to the protein encoded by the IL-Hy1 polynucleotide sequence. Alternatively, agents may be rationally selected or designed. As used herein, an agent is said to be “rationally selected or designed” when the agent is chosen based on the configuration of the particular protein. For example, one skilled in the art can readily adapt currently available procedures to generate peptides, pharmaceutical agents and the like capable of binding to a specific peptide sequence in order to generate rationally designed antipeptide peptides, for example see Hurby et al., Application of Synthetic Peptides: Antisense Peptides,” In Synthetic Peptides, A User's Guide, W. H. Freeman, NY (1992), pp. 289-307, and Kaspczak et al., Biochemistry 28:9230-8 (1989), or pharmaceutical agents, or the like.

In addition to the foregoing, one class of agents of the present invention, as broadly described, can be used to control gene expression through binding to one of the ORFs or EMFs of the present invention. As described above, such agents can be randomly screened or rationally designed/selected. Targeting the ORF or EMF allows a skilled artisan to design sequence specific or element specific agents, modulating the expression of either a single ORF or multiple ORFs, which rely on the same EMF for expression control. One class of DNA binding agents are agents which contain base residues which hybridize or form a triple helix formation by binding to DNA or RNA. Such agents can be based on the classic phosphodiester, ribonucleic acid backbone, or can be a variety of sulfhydryl or polymeric derivatives, which have base attachment capacity.

Agents suitable for use in these methods usually contain 20 to 40 bases and are designed to be complementary to a region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxynucleotides as Antisense Inhibitors of Gene Expression, CRC Press, Boca Raton, Fla. (1988)). Triple helix-formation optimally results in a shut-off of RNA transcription from DNA, while antisense RNA hybridization blocks translation of an mRNA molecule into polypeptide. Both techniques have been demonstrated to be effective in model systems. Information contained in the sequences of the present invention is necessary for the design of an antisense or triple helix oligonucleotide and other DNA binding agents. Agents, which bind to a protein encoded by one of the ORFs of the present invention, can be formulated using known techniques to generate a pharmaceutical composition.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

Three-Dimensional Structural Analysis

The predicted three-dimensional structure of IL-1Hy1, generated by the GeneAtlas™ program (MSI) which includes fold predictions from Fischer and Eisensberg (Protein Science 5: 947-955, 1996) and homology models from Sanchez and Sali (Proc. Natl. Acad. Sci., 95: 13597-13602, 1998), suggests IL-1Hy1 is structurally related to IL-1Ra. This analysis can be used to predict residues potentially involved in receptor binding and other residues important to IL-1Hy1 biological function. The three-dimensional structure of IL-1Hy1 will be useful in developing modulators of IL-1Hy1 activity such as antibodies, small molecules, peptides and derivatives thereof.

The three-dimensional structure of IL-1Hy1 may be generated using the structural coordinates set forth below in Table II. In addition, it is understood in the art that molecules or molecular complexes that are defined by the structural coordinates of Table II include those plus or minus a root mean square deviation from the conserved backbone atoms of those amino acids of 2-12 Δ, preferably not more than about 7 Δ, or more preferably not more than about 5 Δ, or most preferably not more than about 2 Δ.

Identification of receptor binding residues and other residues important to IL-1Hy1 biological function will be useful in discovering drugs which may modulate (i.e. increase or decrease) activity of the IL-1 receptor. Small molecules, antibodies and peptides which associate with one or more, or two or more, or three or more, or four or more, or five or more of the receptor binding residues or with other regions of IL-1Hy1 may modulate IL-1Hy1 activity, e.g., by increasing or decreasing its affinity for the IL-1 receptor. An understanding of the receptor binding residues and associations that occur with these residues will facilitate the development of modulators (including antagonists and agonists) of IL-1Hy1 activity, including receptor binding.

The “receptor binding residues” of IL-1Hy1 refer to the amino acid residues of the IL-1Hy1 molecule which interact with the IL-1 receptor or any other receptor to which IL-1Hy1 binds. These amino acids preferably include Lys12, Leu16, Gly30, His32 and Tyr150 of SEQ ID NO: 3 and other amino acids within 2-12 Δ, preferably within 7 Δ, or more preferably within 5 Δ, that may interact with these listed amino acids and/or contribute to the three-dimensional conformation of these listed amino acids.

The “accessory protein binding residues” of IL-1Hy1 refer to the amino acid residues of the IL-1Hy1 molecule which interact with IL-1 receptor accessory protein. These amino acids preferably include Asp148 of SEQ ID NO: 3 and other amino acids within 2-12 Δ, preferably within 7 Δ, or more preferably within 5 Δ, that may interact with this amino acid and/or contribute to the three-dimensional conformation of this amino acid.

The IL-1Hy1 three-dimensional structure allows for the generation of polypeptide variants or non-peptidyl compounds that mimic the three-dimensional structure of IL-1Hy1. The IL-1Hy1 three-dimensional structure also allows for the identification of desirable sites for mutation to create polypeptide or non-peptidyl variants with similar, increased, decreased or different biological activity compared to wild type IL-1Hy1. Through site-directed mutagenesis, receptor binding residues, accessory protein binding residues or other residues involved in IL-1Hy1 biological function may be mutated to create modulators of IL-1 receptor activity. The mutants may act as antagonists or agonists for the IL-1 receptor. These mutants may be useful in therapeutic compositions directed to modulating the activity of IL-1Hy1 or its receptor. These mutations can be deletions, additions or substitutions of receptor binding residues, accessory protein binding residues or other residues important to IL-1Hy1 biological function. Non-conservative substitutions are expected to be more likely to result in different biological activity compared to wild type IL-1Hy1. For example, mutations may alter the surface charge of IL-1Hy1 which may alter the biological activity of IL-1Hy1. Other mutations may affect the ability of IL-1Hy1 variants (1) to bind to IL-1 receptor (IL-IR) or other receptors to which IL-1Hy1 binds, (2) to bind to IL-IR accessory protein, or (3) ability to activate IL-IR. The effect of various mutations on IL-1Hy1 activity can be modeled in three-dimensional representations on a computer using any of the computer programs described herein.

Molecular modeling may be carried out using, e.g., the structural coordinates described herein, and any computer programs known in the art. For example, programs which predict binding sites and aid in designing modulators based on three-dimensional structural models include, but are not limited to, GRID (Oxford University) which aids in determining energetically favorable binding sites (Goodford, J. Med. Chem. 28: 849-857, 1985), MCSS (Molecular Simulations, Burlington, Mass.) which aids in determining functional maps of binding sites (Miranker and Karplus, Proteins, Structure, Function, and Genetics, 11: 29-34, 1991), AUTODOCK (Scripps Research) which aids in automated docking of substrates to proteins (Goodsell and Olsen, Proteins, Structure, Function, and Genetics, 8: 195-202, 1990, DOCK (University of San Francisco) which aids in determining macromolecular-ligand interactions (Kuntz et al., J. Mol. Biol. 161: 269-288, 1982).

The term “structure coordinates” refers to Cartesian coordinates derived from mathematical equations to generate the three-dimensional model of IL-1Hy1 as modeled dervied from its primary amino acid sequence using, e.g., the GeneAtlas™ program. The model is used to establish the positions of the individual atoms of the IL-1Hy1 protein.

Those of skill in the art understand that a set of structure coordinates for a molecule or a portion thereof is a relative set of points that define a structure in three dimensions. Thus, it is possible that an entirely different set of coordinates could define a similar structure. Moreover, slight variations in the individual coordinates will have little effect on overall shape. Variations in coordinates may be generated by mathematical manipulations of the structural coordinates, e.g., by permutations of the structure coordinates, fractionalization of the structure coordinates, integer additions or subtractions to sets of the structure coordinates, inversion of the structure coordinates or any combination of the above.

Various computational analyses may be done to determine whether a molecule or a portion thereof is sufficiently similar, e.g., using current software applications, such as the Molecular Similarity application of QUANTA (Molecular Simulations Inc., San Diego, Calif.) version 4.1, and as described in the accompanying User's Guide.

The term “root mean square deviation” means the square root of the arithmetic mean of the squares of the deviations from the mean and is a way to express the deviation or variation from a trend or object. For purposes of the invention, the “root mean square deviation” defines the variation in the backbone of a protein from the polypeptide backbone of IL-1Hy1 or a portion thereof or selected residues thereof, as substantially defined by the structural coordinates in Table II below.

The present invention is illustrated in the following examples. Example 1 addresses the detection of IL-1Hy1 protein expression in human tissues by immunohistochemistry. Example 2 demonstrates that diseased tissue has increased plasma cells and an increase in the percentage of plasma cells expressing IL-1Hy1. Example 3 addresses detection of IL-1Hy1 by in situ hybridization with a DNA probe. Example 4 addresses detection of IL-1Hy1 by in situ hybridization with a riboprobe. Example 5 described IL-1Hy1 expression in skin fibroblast cells. Example 6 relates to expression of IL-1Hy1 in monocytic cells. Example 7 addresses IL-1Hy1-induced ICAM-1 expression. Example 8 addresses IL-1Hy1 tolerance and toxicity in animal studies. Example 9 addresses IL-1Hy1 activation of B cell proliferation. Example 10 relates to IL-1Hy1 activation of B cell signaling. Example 11 addresses IL-1Hy1 activation of transcription factors in B cells. Example 12 addresses IL-1Hy1 inhibition of IL-10 induced IgA production. Example 13 addresses IL-1Hy1 activation of superoxide production. Example 14 addresses detection of IL-1Hy1 expression in colonic tissues with immunohisotchemistry. Example 15 addresses that IL-1Hy1 increased the yield of plasma cells in human B cell cultures. Example 16 describes that IL-1Hy1 increased the yield of plasma cells in mouse B splenic cultures. Example 17 addresses IL-1Hy1 stimulation of B cell differentiation in vivo. Example 18 relates to evaluation of IL-1 agonist activity. Example 19 describes that IL-1Hy1 treatment increases total white blood cells, and in particular B cells, in vivo.

Upon consideration of the present disclosure, one of skill in the art will appreciate that many other embodiments and variations may be made in the scope of the present invention. Accordingly, it is intended that the broader aspects of the present invention not be limited to the disclosure of the following examples.

### EXAMPLES

### Example 1

### Detection of IL-1Hy1 Protein Expression in Human Tissues by Immunohistochemistry

Slides of three different human tissue samples (tonsil, skin, and allergic nasal polyps from patients suffering from chronic allergy conditions) were stained with the rabbit polyclonal anti-IL1Hy1 antibody specific for IL-1Hy1 prepared by immunizing rabbits with IL-1Hy peptide: RLTQLPENGGWNA (SEQ ID NO: 8) using conventional methods (see, e.g., Harlow et al, “Antibodies: A Laboratory Manual”, Cold Spring Harbor Laboratories, Cold Spring Harbor, N.Y. (1998)) and control preimmune serum from the immunized rabbits. Anti-IL-1Hy1 antibody binding was detected by biotinylated goat-anti-rabbit secondary antibody followed by streptavidin-HRP detection. To visually detect staining, the slides were treated with the chromogen 3,3;-diaminobenzidine (DAB; a brown stain) and counter stained with hematoxylin (blue nuclear stain). A negative control was stained in the same way in the absence of anti-IL-1Hy1 antibody.

In addition, double label staining was done as follows (see Myers, J. A., Mehta, P., Hunter, A. W., Berstein, S. A., and Erickson, P. A., “Automated Double-Label: Immunohistochemistry”, Journal of Surgical Pathology, 1: 105-113 (1995). Myers, J. A., D'Andrea, M. R., Hunter, A. W., Mehta, P., Berstein, S. A., and Erickson, P. A., “Automated Double-Label: In Situ Hybridization and Immunohistochemistry”, Journal of Surgical Pathology, 1: 191-203 (1995)) The anti-IL1-Hy1 primary antibody straining was done as described immediately above except that fast red was used as the chromogen (a red stain). A second primary antibody described below (specific for cell phenotype markers) was detected using a biotinylated secondary antibody followed by streptavidin-HRP. DAB was used as the chromogen (brown). The slides were counter stained with hematoxylin (a blue nuclear stain) and were visualized on a light microscope.

Results showed that the IL-1Hy1 protein was located in the cytoplasm of skin epithelial cells. In the tonsil, there was positive staining in scattered cells in some germinal centers and epithelial crypts. There was no staining in T-cells (CD45RO marker), B cells (CD20 marker), macrophages (CD68 marker), or in monocytes, macrophages and Langerhans cells (CD14 marker).

Sections of the allergic nasal polyps were double label stained with antibodies against the IL-1Hy1 protein and IgE, and were also double label stained with anti-Il-1 Hy1 and CD138. Tissue sections were then reacted with secondary antibodies and streptavidin-HRP, and treated with the chromogen 3-amino-9-ethylcarbazole (AEC, red stain). In the allergic nasal polyps, IL-1Hy1 protein is expressed in plasma cells (CD138 positive) that are IgE negative. These results suggest that IL-1Hy1 plays a role in modulating allergic reactions in the allergic nasal polyps. Therefore, IL-1Hy1 or antagonists of its activity (e.g. antibodies) may be useful in the treatment of allergic reactions, such as allergic rhinitis and asthma. Effects of IL-1Hy1 or antagonists thereof can be confirmed in any of the allergy animal models described herein or known in the art.

Serial section immunostaining was also performed on human tissues, including normal nasal tissue, chronic infected nasal polyps, allergic nasal polyps, normal lungs, and lung tissue from patients with chronic bronchitis due to chronic infection. Tissue sections were reacted with rabbit polyclonal anti-IL 1-Hy1 antibody prepared as described above. Anti-IL-1Hy1 antibody binding was detected by biotinylated goat-anti-rabbit secondary antibody followed by streptavidin-AP detection. To visually detect staining, the slides were treated with the chromagen, (Fast Red) and counter stained with hematoxylin (blue nuclear stain).

The results demonstrate that allergic nasal polyps and chronically infected nasal polyps had many more cells expressing IL-1Hy1 protein than the normal nasal polyps. Furthermore, the majority of the IL-1Hy1 expressing cells are IgA-producing plasma cells. In the lung tissues tested, chronic bronchitis lung tissues had many more IL-1Hy1 expressing cells than normal lung tissue. The IL-1Hy1 expressing cells included plasma cells, macrophages and bronchial epithelium cells; expression was highest in the plasma cells. These results suggest that IL-1Hy1 expressing cells are recruited to the site of allergic, infected or inflamed tissue and play a role in modulating inflammation due to allergy and/or acute or chronic infection.

Biopsy samples from normal nasal tissue and patients with allergic nasal polyps, chronic infected nasal polyps, normal lung and chronically infected lung were tested for IL-1Hy1 expressing cells (HLC) as described above. In this study, there were more IL-1Hy1 expressing cells and eosinophils in allergic nasal polyps than in normal nasal (p=0.074 for both cell populations) and chronically infected nasal tissues (p=0.07 for HLC, p=0.08 for eosinophils). There were also more IL-1 Hy1 expressing cells and eosinophils in chronic lung tissue than in normal lung tissue (p=0.128 for HLC, p=0.197 for eosinophils), although the difference was less apparent for the eosinophils. With respect to allergic nasal polyp and chronically infected lung tissues, these results were consistent with results observed in the first experiments.

The values of probability (p value based on the Students T test of matched samples), while ranging from 0.07 to 1.97, were not significantly different due to the low sample size (n=3).

### Example 2

### Diseased Tissue Demonstrate Increased Plasma Cell Levels and an Increase in the Percentage of Plasma Cells Expressing IL-1Hy1

Slides of tissue from an allergic nasal polyp, ulecerative colitis, Crohn's disease, cystic fibrosis lung, asthma, and pyelonephritis and the respective normal tissue for each disease were stained with the rabbit polyclonal anti-IL-1Hy1 antibody specific for IL-1Hy1 prepared by immunizing rabbits with IL-1Hy peptide: RLTQLPENGGWNA (SEQ ID NO: 8) using conventional methods (see, e.g., Harlow et al., “Antibodies: A Laboratory Manual”, Cold Spring Harbor Laboratories, Cold Spring Harbor, N.Y. (1998)) and control preimmune serum from the immunized rabbits. Anti-IL-1Hy1 antibody binding was detected by biotinylated goat-anti-rabbit secondary antibody followed by streptavidin-HRP detection. To visually detect staining, the slides were treated with the chromogen 3,3-diaminobenzidine (DAB; a brown stain) and counter stained with hematoxylin (blue nuclear stain). A negative control was stained in the same way in the absence of anti-IL-1Hy1 antibody.

In particular, two specimens of each tissue type were serial sectioned and stained with hematoxylin and eosin, an anti-CD 138 antibody (plasma cell marker) and the anti-IL1Hy1 antibody. Subsequently, the stained cells were visually counted under a light microscope (40×) using an ocular grid, which encompassed 62,500 square microns in each field. For each sample, 16 fields were counted per square millimeter of tissue.

In both diseased and normal tissue, IL-1Hy1 protein was detected mainly in plasma cells. However, the normal samples had fewer plasma cells and a lower percentage of IL-1Hy1 expressing plasma cells. In both disease states, not all plasma cells were positively stained for IL-1Hy1. Furthermore, there was slight staining detected in epithelial cells in all tissues. The results are provided in Table I.

TABLE I
IL-1Hy1 labeled plasma cells as per % of
total plasma cell population

| Disease | Normal | Diseased |
|---|---|---|
| Allergic nasal polyp | 32.7% | 70.5% |
| Ulcerative colitis | 32% | 61.1% |
| Crohn's Disease | 32% | 44.7% |
| Cystic Fibrosis | 31% | 74.7% |
| Asthma | 31% | 63.1% |
| Pyelonephritis | 17% | 92% |

### Example 3

### Detection of IL-1Hy1 Protein Expression in Human Tissues by in-situ Hybridization Using DNA Probe

To determine tissue and cell types that express IL-1Hy1 mRNA, a 985 nucleotide EcoRI and HindIII fragment (which included the complete IL-1Hy1 open reading frame) of the RTA00000273.c.07 clone from a fetal liver-spleen cDNA library was used as a probe on a panel of sectioned human tissues. The probe was labeled using the digoxigenin labeling kit supplied by Boehringer-Mannheim using manufacturer's directions. Automated in-situ hybridization was performed by QualTek Molecular Labs (see Myers, et al., J. Surg. Path., 1:191-203 (1995). All tissues were fixed in 10% neutral buffered formalin, paraffin-embedded and cut into 4 μm sections.

Cells in skin, brain and tonsil specifically hybridized to the IL-1Hy1 probe. A strong signal was detected in the basal layer of the skin epithelia. Sporadic cells in the tonsil also produced a signal with strong intensity. Brain cerebellum tissue provides evidence of expression in presumed infiltrating leukocytes found surrounding an artery in the white matter. A different section of cerebellum from the same individual exhibits staining of presumed glial cells located in the molecular layer.

### Example 4

### Detection of IL-1Hy1 mRNA Expression in Human Tissues by in-situ Hybridization Using Riboprobe

The following three pairs of riboprobes were labeled using the digoxigenin labeling kit from Boehringer-Mannheim following manufacturer's directions.

| | |
|---|---|
| Hy1-RNA1-5 5′-CACAGCTCCCGCCAGGAGAA-3′ | (SEQ ID NO:9) |
| Hy1-RNA1-3 5′-GGGACCACGCTGATCTCTTC-3′ | (SEQ ID NO:10) |
| Hy1-RNA2-5 5′-AGCTTCCCGAGAATGGTGGC-3′ | (SEQ ID NO:11) |
| Hy1-RNA2-3 5′-GTGGTCAGGTGCCCACTAAG-3′ | (SEQ ID NO:12) |
| Hy1-RNA3-5 5′-CTGGGTAAGGAACTTAAAGAAC- | (SEQ ID NO:13) |

3′

| | |
|---|---|
| Hy1-RNA3-3 5′-TCTTAACTAACTACATCTGCA- | (SEQ ID NO:14) |

3′

Serial sections of human normal tonsil were exposed to the DIG-labeled IL-1 Hy1 riboprobes and to antibodies for the following cell phenotype marker proteins: CD20 (B cells), K167 (proliferating cells) CD3 (T cells), CD1a (dendritic and langerhans cells), CD14 (monocytes, macrophages and langerhans cells), CD68 (macrophages), LN5 (macrophages, mantle zone cells and histocytes) and epithelial membrane antigen. Staining was done as described above.

The IL-1Hy1 gene was expressed in scattered cells in some germinal centers (in the area where B cells are activated) and in the epithelial crypts. It was not clear whether it is expressed in macrophages or a subset of activated B cells.

### Example 5

### Expression of IL-1Hy1 in Skin Fibroblast Cells

Western blot analysis was performed to detect IL-1Hy1 polypeptide expression in human skin fibroblasts. Normal human foreskin fibroblast cells (ATCC No. CCD1098) were lysed with PLB lysis buffer, the lysate was separated on a SDS polyacrylamide gel, and the resolved proteins were transferred to a filter as described in Example 16. The filter was probed with a polyclonal anti-IL-1Hy1 antibody specific for IL-1Hy1 as prepared in Example 1.

Results indicated that IL-1Hy1 polypeptide was expressed in human foreskin fibroblasts.

### Example 6

### Expression of IL-1Hy1 in Monocytic Cells

Western blot analysis was performed to detect IL-1Hy1 polypeptide expression in human monocytic cells. Activated human THP-1 cells were lysed with PLB lysis buffer, the lysate was separated on a SDS polyacrylamide gel, and the resolved proteins were transferred to a filter. The filter was probed with a polyclonal anti-IL-1Hy1 antibody specific for IL-1Hy1 as prepared in Example 1.

Results indicated that IL-1Hy1 polypeptide was expressed in THP-1 cells.

### Example 7

### Induction of ICAM-1 Expression by IL-1Hy1

Airway inflammation is characterized by a local influx of neutrophils and increased expression of adhesion molecules such as ICAM-1. To determine if IL-1 Hy1 plays a role in airway inflammation, the effect of IL-1Hy1 on ICAM-1 expression in small airway epithelial cells was examined according to the method described by Tosi et al. (Am. J. Resip. Cell. Mol. Biol., 7: 214-221, 1992).

Small airway epithelial cells (SAEC) were obtained from Clonetics (San Diego, Calif.) and maintained in SAGM media (Clonetics) according to the supplier's instructions. SAEC cells were seeded at 3×10⁵ cells per 60 mm precoated dish. The 60 mm dishes were precoated with a mixture of 10 μg/ml fibronectin (Sigma, St. Louis, Mo.), 30 μg/ml Collagen (commercially known as Vitogen 100, Cohesion, Palo Alto, Calif.), and 10 μg/ml bovine serum albulmin (Sigma).

Twenty four hours after seeding, the cells were washed once with growth media and then treated (i.e., stimulated) with IL-1β at 1 ng/ml and/or IL-1Hy1 at 10-fold, 100-fold, 1000-fold etc, concentrations for 24 hours. IL-1β and IL-1Hy1 were added separately or together as indicated. After the 24 hour incubation, the cells were washed with PBS, treated with 0.5% trypsin, and resuspended in 0.1 ml FACS buffer (3% fetal bovine serum/0.01% azide/PBS). The cells were then incubated with anti-ICAM-1 antibody conjugated with R-phycoeryphrin (PE) (Pharmingen) for 30 minutes. Subsequently, the cells were washed and resuspended in 25 ml FACS buffer for analysis on a fluorescent activated cell sorter.

The FACS analysis indicated that treatment with IL-1β increased ICAM-1 by 130% expression and that IL-1Hy1 synergized with IL-1β to further increase ICAM-1 expression. The presence of ten-fold excess of IL-1Hy1 together with IL-1β increased expression of ICAM-1 expression by 30-40% as compared to IL-1β stimulation alone. Stimulation with IL-1Hy1 alone, however, had variable effects on ICAM-1 expression.

### Example 8

### Tolerance and Toxicity Studies

The maximun tolerated dose (MTD) and acute toxicity of IL-1Hy1 polypeptide was determined in mice. Recombinant human IL-1Hy1 was expressed in E. coli. and purified to homogeneity as described above. The concentration of purified IL-1Hy1 was 24.4 mg/ml and each dose was administered in a 0.2 ml volume of 100 mM sodium chloride and 20 mM sodium phosphate pH 7.0.

For the MTD study, three outbred mice (CDI) weighing approximately 30 g received an initial intravenous dose (150 mg/kg) of the IL-1Hy1 formulation via the tail vein. These mice were observed for several hours and again the next morning and did not exhibit any clinical signs.

For the acute toxicity study, four groups of six outbred mice, weighing approximately 30 g, each received a single intravenous dose of IL-1Hy1 via the tail vein. Each group received a different dose of IL-1Hy1, (vehicle alone, 3.75 mg/kg, 37.5 mg/kg, 150 mg/kg) and were observed daily for seven days. The mice did not exhibit any unusual clinical signs. On day 7, the mice were euthanized and gross necropsy did not indicate any abnormal findings. These results indicated that IL-1 Hy1 appeared to be well tolerated in mice up to a dose of 150 mg/kg.

### Example 9

### IL-1Hy1 Activates Proliferation of B Cells

IL-1Hy1 activation of B cell proliferation was demonstrated on CA46 cells, a Burkitt's lymphoma cell line obtained from the ATCC (accession no. CRL-1648). The CA46 cells were cultured in ATCC medium (RPMI 1640 containing 2 mM L-glutamine adjusted to contain 1.5 g/L sodium bicarbonate, 4.5 g/L glucose, 10 mM HEPES, and 1.0 mM sodium pyruvate; 80%) supplemented with 20% fetal bovine serum (FBS). For each experiment, 1×10⁶ cells were preactivated with 5 μg/ml anti-IgM antibody (Irvine Scientific) for 24 hours at 37° C. in 5% CO₂. After the 14 preincubation, 2×10⁴ cells were plated in 150 μl of ATCC media. The cells were treated with increasing concentration of IL-1Hy1 (5-500 ng/ml) and incubated for 72 hours at 37° C. in 5% CO₂. As a positive control, CA46 cells were treated with 20 ng/ml of IL-10 (R&D Systems). After the incubation, cell proliferation was measured colorimetrically using the Cell Titer assay (Piomega) according to the manufacturer's instructions. The measurements were taken at O.D. 490 after a two-three hour incubation at 37° C. in 5% CO₂.

Treatment with IL-1Hy1 resulted in a significant dose-dependent increase in activated B cell proliferation compared to the untreated control. This data suggests that IL-1Hy1 has IL-1 agonistic function in addition to its established IL-1 antagonist function.

### Example 10

### IL-1Hy1 Activates B Cell Signaling

To determine if IL-1Hy1 expression activated B cell signaling, total tyrosine phosphorylation within Burkitt's lymphoma cells was measured with a commercially available phosphoprotein assay (Bio-Rad, Hercules, Calif.) according to the manufacturer's instructions. To carry out the assay, purified anti-phosphotyrosine antibodies (Cell Signaling Technology, Beverly, Mass.) were conjugated to microspheres (Bio-Rad, Hercules, Calif.) according to the manufacturer's instructions.

CA46 cells (9×10⁵) were stimulated with various concentrations of IL-1Hy1 (0.5 ng/ml, 5 ng/ml, 50 ng/ml) for 20 minutes in ATCC medium. The reaction was terminated by quickly rinsing the cells with ice-cold TBS (Bio-Rad). Subsequently, the cells were incubated with ice-cold Lysis Buffer B (Bio-Rad; Hercules, Calif.) for 20 minutes at 4° C. The lysate was cleared by centrifugation at 2,000 g for 30 minutes.

The clarified lysates were incubated with the anti-phosphotyrosine conjugated microspheres in Assay Buffer B (Bio-Rad; Hercules, Calif.) in a 96-well filter plate (Millipore, Bedford, Mass.) overnight at room temperature with constant shaking. Subsequently, the micorspheres with the immune-complex were washed with TBS containing 0.2% Tween-20 (TBST) and incubated with biotinylated anti-phosphotyrosine antibody in TBST containing 5% mouse serum at room temperature for 30 minutes with constant shaking. After the incubation, the microspheres were washed with TBST and further incubated with 2 μg/ml of streptavidin-PE.

The resulting microspheres with the reaction complex were analyzed using the Luminex Reader (Bio-Rad, Hercules, Calif.). This assay demonstrated that IL-1Hy1 significantly increased total tyrosine phosphorylation in B cells in a dose dependent manner. This data indicates that IL-1Hy1 activated B cell signaling.

### Example 11

### IL-1Hy1 Activates Transcription Factors in B Cells

To further evaluate the IL-1Hy1 activated B cell signaling, the ability of IL-1Hy1 to phosphorylate various transcription factors was analyzed. CA46 cells were incubated with various concentrations of IL-1Hy1 and a phosphoprotien assay (Bio-Rad, Hercules, Calif.) was carried out as described in Example 8. Briefly, the microspheres were coated with purified anti-JNK (BD Bioscience, San Diego, Calif.), anti-p38MAPK (Upstate Biotechnology, Lake Placid, N.Y.), anti-ERK (Santa Cruz Biotechnology, Santa Cruz, Calif.) or anti-STAT (Sigma, St. Louis, Mo.) antibodies. The microsphere sets against the various transcription factors were incubated with CA46 cell lysates. Phosphoylation was detected with biotinylated anti-phospho-JNK antibody (dual phosphorylation form), anti-phospho-p38MAPK antibody (dual phosphorylation form), anti-phospho-ERK antibody (dual phosphorylation form) and anti-phospho-STAT3 antibody (pY704). Treatment of CA46 cells with IL-1Hy1 caused a significant increase in phosphorylation of the transcription factors ERK, STAT3, p38 and JNK. These results demonstrated that IL-1Hy1 activated these transcription factors in B cells.

### Example 12

### IL-1Hy1 Inhibits IL-10 Induced IgA Production

To determine if IL-1Hy1 affects IL-10 induced IgA production, assays on human naive B cells were carried out. Human naive B cells were purified from peripheral blood collected at the Stanford Blood Center according to the Mitenyi Biotec (Auburn, Calif.) purification protocols. Briefly, the samples were separated on a filcol gradient and the peripheral blood mononuclear cells were labeled with anti-CD 19 antibody (Miltenyi Biotec) for positive selection of naïve and memory B cells. Subsequently, depletion with anti-CD27 antibody (Miltenyi Biotec) was used to remove the memory B cell population.

The purified B cells were suspended at 1×10⁶ cells/ml in growth medium (Iscoves' medium supplemented with 50 μg/ml human transferrin, 5 μg/ml bovine insulin, 0.5% BSA, 5×10⁵ M β-mercaptoethanol, 5% FBS and penicillin/streptomycin). The purified B cells were preactivated with 0.01% (v/v) SAC (Calbiochem) for 48 hours at 37° C. with 5% CO₂. Subsequently, the cells were plated at 1×10⁵ cells per well in 150 μl of growth medium containing 10 ng/ml IL-10 (R&D Systems) and various concentrations (5, 50, 500 ng/ml) of IL-1Hy1. After a 6 day incubation at 37° C. with 5% CO₂, the supernatant was harvested and the concentration of IgA within the supernatant was measured by an ELISA assay (Bethyl Laboratories, Montgomery, Tex.) according to the manufacturer's instructions.

Treatment with IL-10 alone caused a significant increase in B cell IgA production. The addition of IL-1Hy1 alone to the B cell cultures had no effect on IgA production. When the B cells were treated with both IL-10 and IL-1Hy1, the IL-10 induced increase in IgA production was significantly inhibited. These results were obtained from about 80% of the experiments carried out.

### Example 13

### IL-1Hy1 Activates Superoxide Production

IL-1Hy1 expression is elevated in plasma cells within allergic nasal polyps, as described in Example 18. Therefore experiments were carried out to determine if IL-1Hy1 affects eosinophil function by measuring eosinophil-produced superoxide levels.

HL60 cells (clone 15) were obtained from the ATCC (accession no. CCL-240) and were maintained in RPMI 1640 supplemented with 10% fetal bovine serum (FBS) at 37° C. with 5% CO₂. The cell were stimulated for 4-6 days with 0.5 mM butyric acid (Sigma Chemicals, St. Louis, Mo.) to induce differentiation. The differentiated cells (2.5-5×10⁵ cell/well) were plated in 100 μl of DMEM supplemented with 2.5% FBS. After attachment overnight, the cells were treated for 16 hours with 100 ng/ml IL-5 (R&D Systems), or 100 ng/ml RANTES (R&D Systems) (positive controls), or 500 ng/ml of IL-1Hy1 or a combination of both IL-5 and IL-1Hy1. After the incubation, the cells were pelleted and then resuspended in superoxide assay medium (Calbiochem). The level of superoxide production was measured by Luminol based chemiluminesence using the Superoxide Detection Kit (Calbiochem) according to the manufacturer's instruction.

IL-1Hy1 alone activated superoxide production in the differentiation HL60 cells. IL-5 is known to induce superoxide production in eosinophils. When IL,-5 and IL-1Hy1 were added in combination, the increase in superoxide production was additive.

In addition, secretory IgA (sIgA) is abundant in allergic nasal polyps and is known to induce activated superoxide production in eosinophils. (Motegi et al., Int. Arch Allergy Immunol. 122 (suppl 1): 25-27, 2000). Therefore, it was of interest to determine whether IL-1Hy1 affects sIgA treated cells. IIL60 (clone 15) cells were cultured as described above and plated in sIgA coated wells. These wells were prepared by diluting sIgA (ICN Pharmaceuticals) in PBS to a concentration of 100 μg/ml. The sIgA containing wells were incubated at 37° C. with 5% CO₂ for 3 hours. The wells were washed with PBS before use.

The stimulated HL60 (clone 15) cells were plated in the sIgA coated wells and treated with IL-1Hy1 (50 or 500 ng/ml) or TNFα (100 ng/ml; positive control). Culturing on the sIgA coated well increased superoxide production in the differentiated IIL60 cells. The addition of IL-1IIy1Hy1 as well as TNFα (positive control), further activated superoxide production in these cells.

The elevation of superoxide production suggests that IL-1Hy1 mediates eosinophil function. This data also suggests that IL-1Hy1 may induce inflammation in tissues with IL-1Hy1 expressing plasma cells, such as allergic nasal polyps.

### Example 14

### Detection of IL-1Hy1 Expression in Plasma Cells of Colonic Tissues

Expression of IL-1Hy1 protein was detected with immunohistochemistry in colonic tissue from normal colons, patients with ulcerative colitis, and patients with Crohn's Disease. The immunohistochemical analysis was carried out by QualTek Molecular Labs (Santa Barbara, Calif.) as described in Example 1. In particular, two specimens of each tissue type were serial sectioned and stained with hematoxylin and eosin, an anti-CD138 antibody (plasma cell marker) and either an anti-IL1Hy1 antibody (see Example 1) or an anti-IgA antibody. Subsequently, the stained cells were visually counted under a light microscope (40×) using an ocular grid which encompassed 62,500 square microns in each field. For each sample, 16 fields were counted per square millimeter of tissue.

IL-1Hy1 protein was detected mainly in plasma cells and the results are summarized in Table II. The normal colon samples had the fewest number of plasma cells and the lowest percentage of IL-1Hy1 and IgA expressing plasma cells. In both disease states, not all plasma cells were positively stained for IL-1Hy1 or IgA expression.

TABLE II

| | Percent PC | Percent Increased | |
|---|---|---|---|
| | Expressing IL- | Compared to | Percent PC |
| Colon Tissue Type | 1Hy1 | Normal | Expressing IgA |
| Normal | 32% | — | — |
| Ulcerative Colitsis | 61% | 74% | 51% |
| Crohn's Disease | 45% | 55% | 72% |

The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims. All references cited within the body of the instant specification are hereby incorporated by reference in their entirety.

### Example 15

### IL-1Hy1 Increases the Yield of Plasma Cells in Human B Cell Cultures

Human lymphocytes (PBMC) were obtained by Ficoll-Hypaque density gradient separation (Current Protocol in Immunology, Chapter 7, John Wily, 1998) of peripheral blood of healthy volunteer donors from Stanford University Blood Center. Naïve B cells from PBMC were further purified by CD19 positive selection. CD19 is expressed on most B cells except plasma cells. Selected CD19-positive cells were then subjected to CD27 negative selection using MACS beads from Miltenyi Biotec (Auburn, Calif.) according to manufacturer's instructions. CD27 is expressed on memory B cells and some T cells. The cells were washed and resuspended in B cell growth medium (RPMI+10% fetal bovine serum). The cells were then primed by pretreatment with anti-IgM antibody (10 μg/ml) at a cell density of one million cells per ml for 24 hr at 37° C. at 5% CO₂. The cells (3.5×10⁵ cells per 200 □1) were then cultured in B cell growth medium with IL-1Hy-1 (50 ng/ml) for 7 days at 37° C. 5% CO₂. Untreated samples (i.e. no IL-1Hy1) were included as the background measurement. After the incubation period, cell pellets were washed and resuspended in FACS buffer (PBS/3% fetal bovine serum/0.02% azide). FITC-conjugated anti-CD38 (Pharmingen, San Diego) was added to the cells and incubated for 30 min on ice. The cells were then washed one time in FACS buffer and resuspended in FACS buffer for FACS analysis.

Incubation of naïve B cells in the presence of IL-1Hy1 increased the yield of CD38-positive cells by 120%, 545%, and 682% over background (i.e., the untreated sample in each experiment) in three cell preparations. Three other preparations of naïve B cells incubated in the presence of IL-1Hy1 showed no change. This discrepancy is likely due to donor to donor variation and is recognized in the art as typical of human blood experiments.

### Example 16

### IL-1Hy1 Increases the Yield of Plasma Cells in Mouse B Splenic Cultures

B cells obtained from mouse spleen where used in the IL-1Hy1 incubation procedure described in Example 14. The same procedure as from humans was followed except for the following: 1) CD43 negative selection was used instead of CD19; 2) priming was done using LPS (50 μg/ml) instead of anti-IgM; and 3) anti-CD138 antibodies were used for staining of plasma cells in the FACS analysis (unlike human plasma cells, mouse plasma cells downregulate CD38 expression). Furthermore, the effect of different doses of IL-1Hy1 was determined from the mouse data.

IL-1Hy1 increased the yield of CD138-positive cells in a dose dependent manner (on average, ˜8% for 5 ng/ml IL-1Hy1; ˜23% for 50 ng/ml IL-1Hy1; ˜38% for 250 ng/ml IL-1Hy1) compared to untreated cultures. The dose effect appeared to plateau at 250 ng/ml. The percent of CD138-positive cells obtained at 250 ng/ml IL-1Hy1 was similar to the percentage obtained when the naïve B cells were incubated in the presence of 20 ng/ml IL-1β.

### Example 17

### IL-1Hy1 to Stimulates B cell Differentiation in vivo

20 ng/ml IL-1Hy1 in saline is injected into liver of neonatal mice; 1 dose per day for five days. After 3 weeks, the mice are bled. Cells are prepared and stained as described in Example 14 and analyzed using flow cytometry. Furthermore, antibody production is determined using ELISA.

### Example 18

### Evaluation of IL-1 Agonist Activity

Assays evaluating B cell activity, differentiation, and proliferation were used to analyze the IL-1 agonist activity of IL-1Hy1 polypeptide. B cell activation was measured in IM-9 B cells (ATCC accession no. CRL-159) by evaluating IgG production. The B cells were stimulated with concentrations of IL-1Hy1 ranging from 5, 50 and 500 ng/ml, and incubated thereafter for 4 days. After the incubation, the conditioned media was harvested and the concentration of IgG produced by the B cells was measured with an ELISA assay. Results in this preliminary experiment indicated no change in IgG production.

### Example 19

### IL-1Hy1 Increased Total White Blood Cells In Vivo

IL-1Hy1 has been shown to stimulate B cell proliferation and signaling in vitro (see Examples 7-9). To determine if IL-1Hy1 effects B cells populations in vivo, B cell adoptive transfer experiments were carried out in rag-2/common gamma double knock out mice (Ly5.2, C57B1/6×C57B1/10 background). These double knock out mice (denoted herein as “recipient mice”) lack the recombinase activating gene 2 and functional receptors for many cytokines, including IL-2, IL-4, IL-7, IL-9 and IL-15 (Cao et al., Immunity 2(3):223-38, 1995; and therefore should not produce NK cells, B cells or T cells.

B cells, for use as donor cells for the adoptive transfer, were isolated from the spleens of B6.5JL mice (Ly5.1, C57B1/6) using magnetic beads (Miltenyi Biotec, Auburn Calif.) specific for CD3, CD4, and CD8 negative selection. The CD3-negaitve, CD4-negative, CD8-negative splenocyte population was further purified with magnetic beads specific for B220+cells. Prior to B cell transfer, the immune systems of the recipient mice were suppressed by treating with busulfan (6.25 mg/kg/day) for 4 days and cyclophosphamide (50 mg/kg/day) for 1 day. Subsequently, approximately 10 to 20 million CD3-CD4-CD8-B220+ cells were transferred into the immunosuppressed recipient mice.

To determine the effect of IL-1Hy1 on the total number of white blood cells (WBC) in vivo, recipient mice (N=5) were injected with 500 mg/kg/day of IL-1Hy1, IL-4 or PBS for seven days. Subsequently, the mice were bled and the total number of WBC (neutrophils, lymphocytes, monocytes, basophils and eosinophils) in the peripheral blood were measured with a CellDyn 3700 hematology analyzer according to the manufacturer's instructions. The total number of WBC in the peripheral blood increased after 7 days of IL-1Hy1 injections as compared to the PBS-treated mice (40-80% increase) while IL-4 injection had no effect on the total number of WBC.

To determine the effect of IL-1Hy1 on the percentage of B220+ cells in the spleen, recipient mice (N=2) were injected with 500 mg/kg/day of IL-Hy1, heat-inactivated IL-1Hy1 or PBS for six days. Subsequently, the mice were sacrificed and the spleen cells were analyzed by flow cytometry analysis using fluorescence-conjugated monoclonal antibodies specific for Ly5.1, B220, CD138 and CD4/CD8 (BD Phaminogen, San Diego, Calif.) The percentage of B220+ cells doubled in the spleens of IL-1Hy1-treated mice as compared to PBS-treated mice, while treatment with heat-inactivated IL-1Hy1 had no effect on the percentage of B220+ cells in the spleen.

To confirm the above results, the total number of white blood cells in the peripheral blood and spleen were analyzed in additional recipient mice as described above. After six days of IL-1Hy1 injections, the total number of WBC increased in peripheral blood and spleens of IL-1Hy1-treated mice as compared to those treated with PBS, while injection of heat-inactivated IL-1Hy1 had no effect.

The results of the above experiments demonstrate that the total white blood cells and the percentage of B cells increased after IL-1Hy1 treatment in vivo. The in vivo results substantiate the in vitro results described in Examples 7-9, which demonstrate IL-1Hy1 stimulates B cell proliferation and B cell signaling. Therefore, IL-1Hy1 may serve as a growth or differentiation factor for hematopoietic cells.

1. A method of inhibiting B cell proliferation, activation or differentiation comprising administering an inhibitor of IL-1Hy1 activity to a human with elevated B cell levels or B cell activity, in an amount effective to inhibit B cell proliferation induced by IL-1Hy1 of SEQ ID NO: 3.

2. The method of claim 1 wherein the inhibitor is an antibody.

3. The method of claim 2 wherein the antibody is a humanized antibody.

4. The method of claim 2 wherein the antibody is a monoclonal antibody.

5. The method of claim 1 wherein the inhibitor is a polynucleotide that binds to SEQ ID NO: 2 or its complement and inhibits IL-1Hy1 polypeptide production.

6. The method of claim 1 wherein said human is suffering from a B cell lymphoproliferative disease.

7. The method of claim 6 wherein said human is suffering from lymphoma, leukemia or myeloma.

8. The method of claim 6 wherein said human is suffering from an autoimmune disease.

9. The method of claim 5 wherein said human is suffering from an allergy, asthma or allergic rhinitis.

10. Use of an inhibitor of IL-1Hy1 activity in preparation of a medicament for use in reducing B cell proliferation, activation or differentiation.

11 A composition comprising an inhibitor of IL-1Hy1 activity in an amount effective to inhibit B cell proliferation, B cell differention or B cell activity induced by IL-1Hy1.

12. A method of stimulating B cell proliferation comprising administering an effective amount of IL-1Hy1, comprising the amino acid sequence of SEQ ID NO: 3, to a human in need of higher B cell levels or activity.

13. The method of claim 12 wherein IL-1Hy1 is concurrently administered with a vaccine to improve efficacy of the vaccine.

14. The method of claim 12 wherein said human is suffering from a B cell deficiency.

15. The method of claim 12 wherein said human is suffering from an infection.

16. Use of IL-1Hy1 polypeptide comprising the amino acid sequence of SEQ ID NO: 3 in an amount effective to stimulate proliferation, differention or activtion of B cells in preparation of a medicament for use in stimulating B cell proliferation, differentation or activation.

17. A composition comprising IL-1Hy1 polypeptide comprising the amino acid sequence of SEQ ID NO: 3 in an amount effective to stimulate B cell proliferation, differentiation or activation.

18. A method of treating an IgA related autoimmune disease comprising the steps of administering a therapeutically effective amount of IL-1Hy1, comprising the amino acid sequence of SEQ ID NO: 3, to a human suffering from an disorder related to elevated IgA levels.

19. The method of claim 18 wherein IL-1Hy1 is concurrently administered with a vaccine to improve efficacy of the vaccine.

20. Use of an IL-1Hy1 polypeptide comprising the amino acid sequence of SEQ ID NO: 3 in an amount effective to reduce IgA production in preparation of a medicament for use in reducing IgA production.

21. A composition comprising IL-1Hy1 polypeptide comprising the amino acid sequence of SEQ ID NO: 3 in an amount effective to reduce IgA production.

22. A method of stimulating B-cell differentiation comprising contacting a B-cell with an amount of IL-1Hy1 of SEQ ID NO: 3 effective to stimulate B-cell differentiation.

23. The method of claim 22, wherein the contacting comprises culturing the B-cell at a first stage of B-cell development in a growth medium comprising IL-1Hy1.

24. The method of claim 22, wherein the composition comprises recombinant IL-1Hy1.

25. The method of claim 22, wherein the B-cell is a naïve B cell.

26. The method of claim 22, wherein the B-cell development is stimulated to secrete antibodies.

27. The method of claim 22, wherein the B-cell is a human lymphocyte.

28. The method of claim 22, wherein the lymphocyte is a peripheral blood mononuclear cell (PBMC).

29. A method of treating a patient suffering from a B-cell related disorder by administering a pharmaceutical composition comprising IL-1Hy1 in an amount effective to stimulate B-cell differentiation.

30. The method of claim 29 wherein the composition is concurrently administered with a vaccine to improve efficacy of the vaccine.

31. The method of claim 29, wherein in the patient is suffering from an infection.

32. The method of claim 29, wherein the patient is suffering from an immunogloblulin deficiency syndrome.

33. The method of claim 29, wherein the immunoglobulin deficiency syndrome is agammagglobilinemia.

34. A method of inhibiting B-cell differentiation comprising contacting a B-cell with an inhibitor of IL1-Hy1 activity in an amount effective to B-cell differentiation.

35. The method of claim 34 wherein the inhibitor is an antibody.

36. A method of screening for compounds that modulate B-cell differentiation, the method comprising the steps of:
(a) incubating, with and without a test compound, a population of cells containing one or more cells at a first stage of B-cell development with a composition comprising an amount of IL-1Hy1 activity effective to stimulate B-cell differentiation; and
(b) determining an effect of the test compound on the ability of IL-1Hy1 to stimulate B cell differentiation, wherein an alteration of the ability of IL-1Hy1 to stimulate B cell differentiation indicates a modulator of B-cell differentiation.

37. The method of claim 36, wherein the effect an increase in the ability of IL-1Hy1 to stimulate B cell differentiation.

38. The method of claim 36, wherein the effect is a decrease in the ability of IL-1Hy1 to stimulate B cell differentiation.
